(19) United States
(12) Patent Application Publication
(10) Pub. No.: US 2012/0252820 A1
(43) Pub. Date: Oct. 4, 2012

(54) METHODS OF TREATING FUNGAL INFECTIONS

(75) Inventors: Kim Lewis, Newton, MA (US); Michael David Lafleur, Braintree, MA (US)

(73) Assignee: NORTHEASTERN UNIVERSITY, Boston, MA (US)

(21) Appl. No.: 13/513,149

(22) PCT Filed: Apr. 22, 2009
(86) PCT No.: PCT/US2009/041403

§ 371 Date: May 31, 2012

Related U.S. Application Data
(60) Provisional application No. 61/046,953, filed on Apr. 22, 2008.

(51) Int. Cl. A61K 31/13; A01N 43/50; A01N 33/04; A61K 31/4015; A01N 43/36; A61K 31/495; A01N 43/60; A61K 31/16; A01N 37/18; A61K 31/18; A01N 41/06; A61K 31/045; A01N 31/06; A01P 3/00; A61P 31/10; A61K 31/4174

(52) U.S. Cl. 514/255.03; 514/399; 514/662; 514/396; 514/424; 514/629; 514/604; 514/729

(57) ABSTRACT

Methods of identifying compounds that potentiate the activity of antifungal agents, potentiators identified by these methods, and methods of using potentiators to treat fungal infections are disclosed.

### CROSS REFERENCE TO RELATED APPLICATIONS

This application claims priority from U.S. Provisional Application Ser. No. 61/046,953, filed on Apr. 22, 2008, the contents of which are hereby incorporated by reference in their entirety herein.

### FIELD OF THE INVENTION

The invention relates to medicine, and more particularly to the treatment of fungal infections.

### BACKGROUND OF THE INVENTION

Multidrug tolerance of pathogens is in large part the result of the entry of microbial cells into a dormant state. Such dormant cells can be responsible for latent (chronic) diseases or relapsing disorders. Many such dormant cells can be suppressed by known antifungals but have not been eradicated.

Fungal biofilms are communities of cells that settle and proliferate on surfaces and are covered by an exopolymer matrix. They are slow-growing and many are in the stationary phase of growth. They can be formed by most, if not all, pathogens. According to the CDC, 65% of all infections in the United States are caused by biofilms that can be formed by common pathogens. The biofilm exopolymer matrix protects against immune cells, and persister cells that are contained in the biofilm can survive both the onslaught of antifungal treatment and the immune system. When antifungal levels decrease, these persister cells can repopulate the biofilm, which will shed off new planktonic cells, producing a relapsing biofilm infection. Fungal biofilm infections are highly recalcitrant to antifungal treatment. Therefore, there is a need for adequate therapy against these infections.

### SUMMARY OF THE INVENTION

Aspects of the invention are based, at least in part, on the identification of compounds that can inhibit the growth of, or kill, a fungus. Accordingly, in one aspect, the invention features a method of inhibiting the growth of, or killing, a fungus, the method comprising contacting the fungus with (i) an antifungal agent, and (ii) a potentiator compound of Formula I:

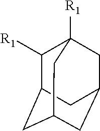

or a pharmaceutically acceptable salt, hydrate, solvate, or prodrug thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby inhibiting the growth of, or killing, the fungus.

In some embodiments, the compounds of Formula I are of the Formula Ia:

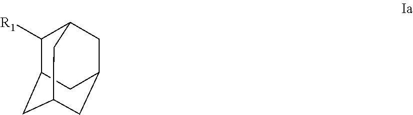

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl.

In some embodiments, R₁ is NH₂.

In other embodiments, R₁ is OH,

In still other embodiments, R₁ is —NHalkyl.

In still other embodiments, R₁ is —N(alkyl)₂.

In a further embodiment, R₁ is a 5- or 6-membered heterocycle.

In another embodiment, R₁ is a 5-membered heterocycle substituted with a carbonyl.

In some embodiments, the compounds of Formula I are of the Formula Ib:

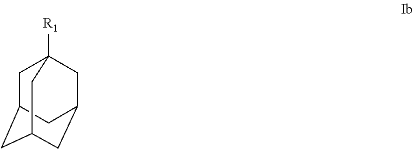

and pharmaceutically acceptable salts, hydrates, solvates, and prodrugs thereof,

wherein each R₁ is independently —OH, —OC(O)H, —OC(O)alkyl, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or alkyl, optionally substituted with alkyl, halogen, OH, or NH₂.

In some embodiments, R₁ is NH₂.

In other embodiments, R₁ is an alkyl, optionally substituted with alkyl, halogen, OH, or NH₂.

In another embodiment, R₁ is an alkyl substituted with NH₂.

In some embodiments, the potentiator compound potentiates the activity of the antifungal agent. In some embodiments, the potentiator compound is not an antifungal compound.

In certain embodiments, the fungus is one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In certain embodiments, the antifungal agent is Amphotericin B, an imidazole (e.g., miconazole), clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or cielopirox.

In another aspect, the invention features a method of treating a fungal infection in a subject in need thereof, the method comprising administering to the subject an effective amount of an antifungal agent in combination with an effective amount of a potentiator compound of Formula I:

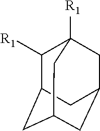

or a pharmaceutically acceptable salt, hydrate, solvate, or prodrug thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby treating the fungal infection.

In some embodiments, the compounds of Formula I are of the Formula Ia:

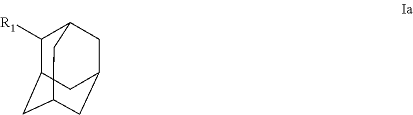

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl.

In some embodiments, R₁ is NH₂.

In other embodiments, R₁ is OH,

In still other embodiments, R₁ is —NHalkyl.

In still other embodiments, R₁ is —N(all 1)₂.

In a further embodiment, R₁ is a 5- or 6-membered heterocycle.

In another embodiment, R₁ is a 5-membered heterocycle substituted with a carbonyl

In some embodiments, the compounds of Formula. I are of the Formula Ib:

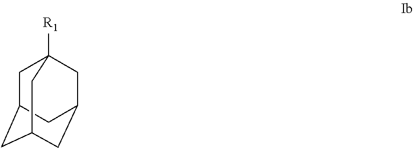

and pharmaceutically acceptable salts, hydrates, solvates, and prodrugs thereof,

wherein each R₁ is independently —OH, —OC(O)H, —OC(O)alkyl, NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, or carbonyl, or alkyl, optionally substituted with alkyl, halogen, OH, or NH₂.

In some embodiments, R₁ is NH₂.

In other embodiments, R₁ is an alkyl, optionally substituted with alkyl, halogen, OH, or NH₂.

In another embodiment, R₁ is an alkyl substituted with NH₂.

In some embodiments, the potentiator compound potentiates the activity of the antifungal agent. In some embodiments, the potentiator compound is not an antifungal compound.

In certain embodiments, the fungal infection comprises one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondi); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In certain embodiments, the antifungal agent is Amphotericin B, an imidazole (e.g., miconazole), clotrimazole, fluconazole, itraconazole, ketoconazole, ravuconazole, posaconazole, voriconazole, caspofungin, micafungin, FK463, anidulafungin (LY303366), hydroxystilbamidine, 5-fluorocytosine, flucytosine, iodide, terbinafine, Nystatin, griseofulvin, or ciclopirox.

In some embodiments, the fungal infection is aspergillosis, blastomycosis, candidiasis (e.g., oral thrush or vaginitis), coccidioidomycosis, cryptococcosis, histoplasmosis, paracoccidiomycosis, sporotrichosis, or zygomycosis. In some embodiments, the fungal infection is associated with a catheter, an orthopedic prostheses, or a heart valve.

In another aspect, the invention features a method of treating relapsing vaginitis in a subject, the method comprising administering to the subject an effective amount of miconazole in combination with an effective amount of potentiator compound of Formula I:

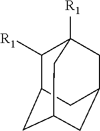

or a pharmaceutically acceptable salt, hydrate, solvate, or prodrug thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby treating the relapsing vaginitis in the subject. In some embodiments, the relapsing vaginitis comprises Candida albicans. In other embodiments, the relapsing vaginitis comprises Candida albicans persister cells.

In some embodiments, the compounds of Formula I are of the Formula Ia:

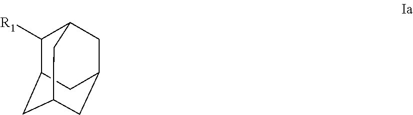

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl.

In some embodiments, R₁ is NH₂.

In other embodiments, R₁ is OH,

In still other embodiments, R₁ is —NHalkyl.

In still other embodiments, R₁ is —N(alkyl)₂.

In a further embodiment, R₁ is a 5- or 6-membered heterocycle.

In another embodiment, R₁ is a 5-membered heterocycle substituted with a carbonyl.

In some embodiments, the compounds of Formula I are of the Formula Ib:

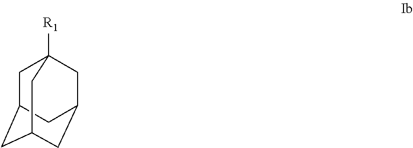

and pharmaceutically acceptable salts, hydrates, solvates, and prodrugs thereof,

wherein each R₁ is independently —OH, —OC(O)H, —OC(O)alkyl, —N₂—NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or alkyl, optionally substituted with alkyl, halogen, OH, or NH₂.

In some embodiments, R₁ is NH₂.

In other embodiments, R₁ is an alkyl, optionally substituted with alkyl, halogen, OH, or NH₂.

In another embodiment, R₁ is an alkyl substituted with NH₂.

In another aspect, the invention features a method of inhibiting the growth of, or killing, a fungus, the method comprising contacting the fungus with a potentiator compound of Formula I:

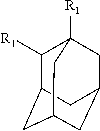

or a pharmaceutically acceptable salt, hydrate, solvate, or prodrug thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby inhibiting the growth of, or killing, the fungus.

In some embodiments, the compounds of Formula I are of the Formula Ia:

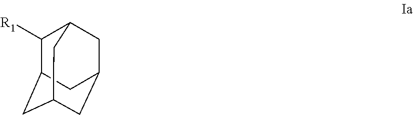

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl.

In some embodiments, R₁ is NH₂.

In other embodiments, R₁ is OH,

In still other embodiments, R₁ is —NHalkyl.

In still other embodiments, R₁ is N(alkyl)₂.

In a further embodiment, R₁ is a 5- or 6-membered heterocycle.

In another embodiment, R₁ is a 5-membered heterocycle substituted with a carbonyl.

In some embodiments, the compounds of Formula I are of the Formula Ib:

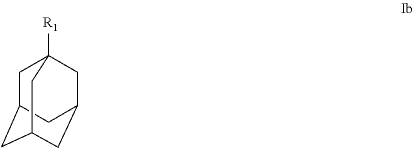

and pharmaceutically acceptable salts, hydrates, solvates, and prodrugs thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or alkyl, optionally substituted with alkyl, halogen, OH, or NH₂.

In some embodiments, R₁ is NH₂.

In other embodiments, R₁ is an alkyl, optionally substituted with alkyl, halogen, OH, or NH₂.

In another embodiment, R₁ is an alkyl substituted with NH₂.

In certain embodiments, the fungus is one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In another aspect, the invention features a method of treating a fungal infection in a subject in need thereof, the method comprising administering to the subject an effective amount of a potentiator compound of Formula I:

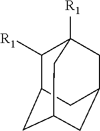

or a pharmaceutically acceptable salt, hydrate, solvate, or prodrug thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby treating the fungal infection.

In some embodiments, the compounds of Formula I are of the Formula Ia:

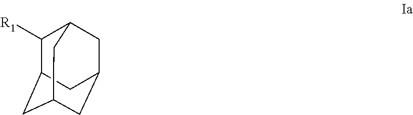

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen. OH, or NH₂, or 5- or 6-membered heterocycle, optionally substituted with alkyl, halogen, OH, NH₂, or carbonyl.

In some embodiments, R₁ is NH₂.

In other embodiments, R₁ is OH,

In still other embodiments, R₁ is —NHalkyl.

In still other embodiments, R₁ is —N(alkyl)₂.

In a further embodiment, R₁ is a 5- or 6-membered heterocycle.

In another embodiment, R₁ is a 5-membered heterocycle substituted with a carbonyl.

In some embodiments, the compounds of Formula I are of the Formula Ib:

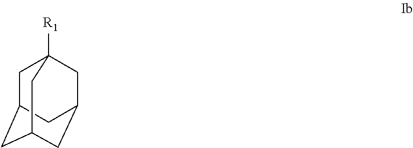

and pharmaceutically acceptable salts, hydrates, solvates, and prodrugs thereof,

wherein each R₁ is independently —OH, —OC(O)H, —OC(O)alkyl, —NH₂, —NH-alkyl, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or alkyl, optionally substituted with alkyl, halogen, OH, or NH₂.

In some embodiments. R₁ is NH₂.

In other embodiments, R₁ is an alkyl, optionally substituted with alkyl, halogen, OH, or NH₂.

In another embodiment, R₁ is an alkyl substituted with NH₂.

In certain embodiments, the fungal infection comprises one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

In some embodiments, the fungus is a recalcitrant fungus. In other embodiments, the fungus is a fungal biofilm. In yet other embodiments, the fungus comprises persister cells.

In some embodiments, the fungal infection is aspergillosis, blastomycosis, candidiasis (e.g., oral thrush or vaginitis), coccidioidomycosis, cryptococcosis, histoplasmosis, paracoccidiomycosis, sporotrichosis, or zygomycosis. In some embodiments, the fungal infection is associated with a catheter, an orthopedic prostheses, or a heart valve.

In another aspect, the invention features a method of treating relapsing vaginitis in a subject, the method comprising administering to the subject an effective amount of miconazole in combination with an effective amount of potentiator compound of Formula I:

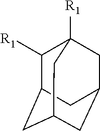

or a pharmaceutically acceptable salt, hydrate, solvate, or prodrug thereof,

wherein each R₁ is independently —OH, —OC(O)H, —OC(O)allyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby treating the relapsing vaginitis in the subject. In some embodiments, the relapsing vaginitis comprises Candida albicans. In other embodiments, the relapsing vaginitis comprises Candida albicans persister cells.

In some embodiments, the compounds of Formula I are of the Formula Ia:

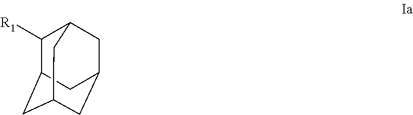

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl.

In some embodiments, R₁ is NH₂.

In other embodiments, R₁ is OH,

In still other embodiments, R₁ is —NHalkyl.

In still other embodiments, R₁ is —N(alkyl)₂.

In a further embodiment, R₁ is a 5- or 6-membered heterocycle.

In another embodiment, R₁ is a 5-membered heterocycle substituted with a carbonyl.

In some embodiments, the compounds of Formula I are of the Formula Ib:

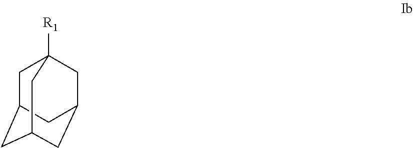

and pharmaceutically acceptable salts, hydrates, solvates, and prodrugs thereof,

wherein each R₁ is independently —OH, —OC(O)H, —OC(O)allyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or alkyl, optionally substituted with alkyl, halogen, OH, or NH₂.

In some embodiments, R₁ is NH₂.

In other embodiments, R₁ is an alkyl, optionally substituted with alkyl, halogen, OH, or NH₂.

In another embodiment, R₁ is an alkyl substituted with NH₂.

In another aspect, the invention features a method of treating or preventing oral candidiasis in a subject, the method comprising administering to the subject an effective amount of miconazole in combination with an effective amount of potentiator compound of Formula I:

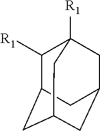

or a pharmaceutically acceptable salt, hydrate, solvate, or prodrug thereof,

wherein each R₁ is independently —OH, —OC(O)H, —OC(O)alkyl, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby treating or preventing the oral candidiasis in the subject. In some embodiments, the oral candidiasis comprises Candida albicans. In other embodiments, the oral candidiasis comprises Candida albicans persister cells.

In some embodiments, the compounds of Formula I are of the Formula Ia:

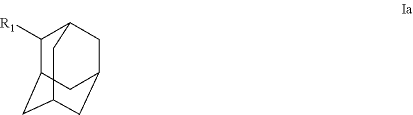

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl.

In some embodiments, R₁ is NH₂.

In other embodiments, R₁ is OH,

In still other embodiments, R₁ is —NHalkyl.

In still other embodiments, R₁ is —N(alkyl)₂.

In a further embodiment, R₁ is a 5- or 6-membered heterocycle.

In another embodiment, R₁ is a 5-membered heterocycle substituted with a carbonyl.

In some embodiments, the compounds of Formula I are of the Formula Ib:

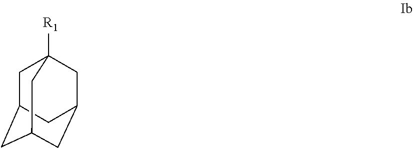

and pharmaceutically acceptable salts, hydrates, solvates, and prodrugs thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂) aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or alkyl, optionally substituted with alkyl, halogen, OH, or

In some embodiments, R₁ is NH₂.

In other embodiments, R₁ is an alkyl, optionally substituted with alkyl, halogen, OH, or NH₂.

In another embodiment, R₁ is an alkyl substituted with NH₂.

In another aspect, the invention features a method of treating a fungal infection of a medical device, the method comprising administering to the subject an effective amount of miconazole in combination with an effective amount of potentiator compound of Formula I:

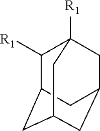

or a pharmaceutically acceptable salt, hydrate, solvate, or prodrug thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby treating the fungal infection of the device. In some embodiments, the infection comprises Candida albicans. In other embodiments, the infection comprises Candida albicans persister cells.

In some embodiments, the medical device is a catheter, an orthopedic prostheses, or a heart valve.

In some embodiments, the compounds of Formula I are of the Formula Ia:

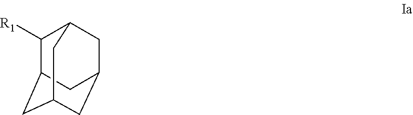

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl.

In some embodiments, R₁ is NH₂.

In other embodiments, R₁ is OH,

In still other embodiments, R₁ is —NHalkyl.

In still other embodiments, R₁ is —N(alkyl)₂.

In a further embodiment, R₁ is a 5- or 6-membered heterocycle.

In another embodiment, R₁ is a 5-membered heterocycle substituted with a carbonyl.

In some embodiments, the compounds of Formula I are of the Formula Ib:

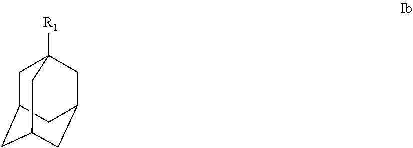

and pharmaceutically acceptable salts, hydrates, solvates, and prodrugs thereof,

wherein each R₁ is independently —OH, —OC(O)H, —OC(O)alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or alkyl, optionally substituted with alkyl, halogen, OH, or NH₂.

In some embodiments, R₁ is NH₂.

In other embodiments, R₁ is an alkyl, optionally substituted with alkyl, halogen, OH, or NH₂.

In another embodiment, R₁ is an alkyl substituted with NH₂.

In another aspect, the invention features a method of inhibiting the growth of, or killing, a C. albicans fungus, the method comprising contacting the fungus with an effective amount of (i) an antifungal agent; and (ii) one or more potentiator compounds of Formula I:

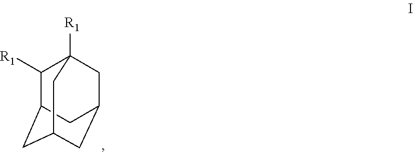

or pharmaceutically acceptable salts, hydrates, and solvates thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby inhibiting the growth of, or killing, the fungus.

In some embodiments, the compounds of Formula I are of the Formula Ia:

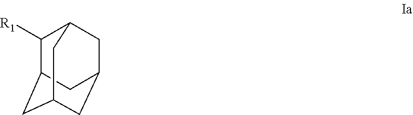

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl.

In some embodiments. R₁ is NH₂.

In other embodiments, R₁ is OH,

In still other embodiments, R₁ is —NHalkyl.

In still other embodiments, R₁ is —N(alkyl)₂.

In a further embodiment, R₁ is a 5- or 6-membered heterocycle.

In another embodiment, R₁ is a 5-membered heterocycle substituted with a carbonyl.

In some embodiments, the compounds of Formula I are of the Formula Ib:

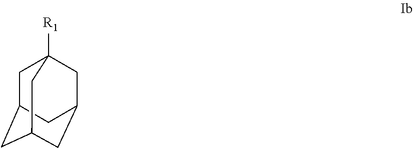

and pharmaceutically acceptable salts, hydrates, solvates, and prodrugs thereof,

wherein each R₁ is independently —OH, —OC(O)H, —OC(O)alkyl, —NH₂, —NH-alkyl, —N (alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or alkyl, optionally substituted with alkyl, halogen, OH, or NH₂.

In some embodiments, R₁ is NH₂.

In other embodiments, R₁ is an alkyl, optionally substituted with alkyl, halogen, OH, or NH₂.

In another embodiment, R₁ is an alkyl substituted with NH₂.

In another aspect, the invention features a method of treating a C. albicans fungal infection in a subject in need thereof, the method comprising administering to the subject an effective amount of (i) an antifungal agent, in combination with (ii) an effective amount of one or more potentiator compounds of Formula I:

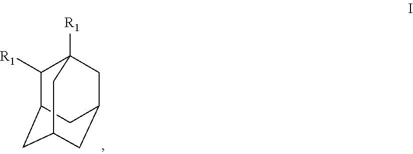

or pharmaceutically acceptable salts, hydrates, and solvates thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or an alkyl, optionally substituted with alkyl, halogen, OH, or NH₂,

thereby treating the fungal infection.

In some embodiments, the compounds of Formula I are of the Formula Ia:

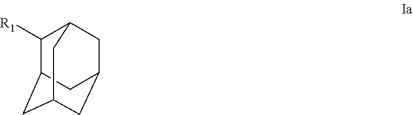

and pharmaceutically acceptable salts, hydrates, solvates, and prodrugs thereof,

wherein R₁ is —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl.

In some embodiments, R₁ is NH₂.

In other embodiments, R₁ is OH,

In still other embodiments, R₁ is —NHalkyl.

In still other embodiments, R₁ is —N(alkyl)₂.

In a further embodiment, R₁ is a 5- or 6-membered heterocycle.

In another embodiment, R₁ is a 5-membered heterocycle substituted with a carbonyl.

In some embodiments, the compounds of Formula I are of the Formula Ib:

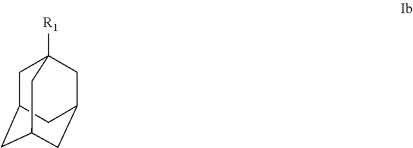

and pharmaceutically acceptable salts, hydrates, solvates, and prodrugs thereof,

wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl, wherein the aryl is optionally substituted, or a 5-membered heteroaryl, optionally substituted with alkyl, halogen, OH, or NH₂, or 5- or 6-membered heterocycle, optionally substituted with alkyl, halogen, OH, NH₂, or carbonyl, or alkyl, optionally substituted with alkyl, halogen, OH, or NH₂.

In some embodiments, R₁ is NH₂.

In other embodiments, R₁ is an alkyl, optionally substituted with alkyl, halogen, OH, or NH₂.

In another embodiment, R₁ is an alkyl substituted with NH₂.

The following figures are presented for the purpose of illustration only, and are not intended to be limiting.

### BRIEF DESCRIPTION OF THE DRAWINGS

FIG. 1A is a graphic representation of the number of surviving C. albicans 3153A cells after treatment with amphotericin B.

FIG. 1B is a graph of the number of surviving C. albicans 3153A cells after treatment with chlorhexidine.

FIG. 2 is a graphic representation of the number of surviving cells following treatment with amphotericin B or chlorhexidine.

FIG. 3 is a graphic representation of the number of surviving cells following treatment with amphotericin B, chlorhexidine, or a combination of amphotericin B and chlorhexidine.

FIG. 4A is a digital representation of a micrograph of live C. albicans planktonic cells.

FIG. 4B is a digital representation of a micrograph of dead C. albicans planktonic cells after treatment with amphotericin B.

FIG. 4C is a digital representation of a micrograph of an untreated C. albicans biofilm.

FIG. 4D is a digital representation of a micrograph of a C. albicans biofilm treated with amphotericin B for 18 hrs.

FIG. 4E is a digital representation of a micrograph of a C. albicans biofilm treated with amphotericin B for 48 hrs.

FIG. 5 is a schematic of a method of screening biofilms for potentiators of miconazole.

FIG. 6 is a graphic representation of a HTS for miconazole potentiator compounds in C. albicans biofilms.

FIG. 7 is a graphic representation of the killing of C. albicans biofilms by increasing concentrations of AC17 alone or in combination with miconazole.

FIG. 8A is a graphic representation of C. albicans clinical isolates treated with amphotericin B or chlorhexidine.

FIG. 8B is a graphic representation of C. albicans biofilms from hip strains either untreated or treated with AC17, miconazole, or a combination of AC17 and miconazole.

FIG. 9A is a graphic representation of the effect of AC17 on biofilm formation from C. albicans cellular suspensions.

FIG. 9B is a graphic representation of the growth curve of AC17 treated or untreated C. albicans cultures.

FIG. 10A is a representation of a micrograph of untreated C. albicans cells.

FIG. 10B is a representation of a micrograph of C. albicans cells treated with AC17.

FIG. 10C is a graphic representation of hyphal length of C. albicans cells untreated or treated with increasing concentrations of AC17.

FIG. 11 depicts representations of micrographs of C. albicans cells grown on various media (Spider, Lee's, YPS, YPD) in the absence or presence of AC17.

FIG. 12A depicts representations of micrographs of wild type C. albicans cells grown in the absence or presence of AC17.

FIG. 12B depicts representations of micrographs of UZ24 C. albicans cells grown in the absence or presence of AC17.

FIG. 12C depicts representations of micrographs of UZ43 C. albicans cells grown in the absence or presence of AC17.

FIG. 12D depicts representations of micrographs of UZ149 C. albicans cells grown in the absence or presence of AC17.

FIG. 13A is a representation of a micrograph of C. albicans strain UZ149 cells grown in YPD medium at 37° C.

FIG. 13B is a representation of a micrograph of C. albicans strain UZ149 cells grown in the presence of doxycycline in YPD medium at 37° C.

FIG. 13C is a representation of a micrograph of C. albicans strain UZ149 cells grown in YPD medium at 37° C. after being diluted 1:500.

FIG. 13D is a representation of a micrograph of C. albicans strain UZ149 cells grown in YPD medium at 37° C. initially in the presence of doxycycline followed by removal of the doxycycline.

FIG. 13E is a representation of a micrograph of C. albicans strain UZ149 cells grown in the presence of doxycycline and AC17 in YPD medium at 37° C.

### DETAILED DESCRIPTION OF THE INVENTION

This application relates, at least in part, to the identification of anti-fungal compounds using screening methods, and the use of such compounds to treat fungal infections.

### DEFINITIONS

The compounds of this disclosure include any and all possible isomers, stereoisomers, enantiomers, diastereomers, tautomers, pharmaceutically acceptable salts, and solvates thereof. Thus, the terms “compound” and “compounds” as used in this disclosure refer to the compounds of this disclosure and any and all possible isomers, stereoisomers, enantiomers, diastereomers, tautomers, pharmaceutically acceptable salts, and solvates thereof.

In general, the compositions of the disclosure can be alternately formulated to comprise, consist of or consist essentially of any appropriate components disclosed in this disclosure. The compositions of the disclosure can additionally, or alternatively, be formulated so as to be devoid, or substantially free, of any components, materials, ingredients, adjuvants or species used in the prior art compositions or that are otherwise not necessary to the achievement of the function and/or objectives of the present disclosure.

The articles “a” and “an” are used in this disclosure to refer to one or more than one (i.e., to at least one) of the grammatical object of the article. By way of example, “an element” means one element or more than one element.

The term “or” is used in this disclosure to mean, and is used interchangeably with, the term “and/or,” unless indicated otherwise.

The term “about” is used in this disclosure to mean a value − or +20% of a given numerical value. Thus, “about 60%” means a value between 60-20% of 60 and 60+20% of 60 (i.e., between 48% and 72%).

The terms “alkyl” and “alk”, unless otherwise specifically defined, refer to a straight or branched chain alkane (hydrocarbon) radical, which may be fully saturated, mono- or polyunsaturated, and can include divalent radicals, having from 1 to about 15 carbon atoms. Examples of saturated hydrocarbon radicals include, but are not limited to, groups such as methyl (Me), ethyl (Et), n-propyl, isopropyl, n-butyl, t-butyl, isobutyl, sec-butyl, homologs and isomers of for example, n-pentyl, n-hexyl, n-heptyl, n-octyl, 1,1-dimethyl-heptyl, 1,2-dimethyl-heptyl, and the like. An unsaturated alkyl group includes one or more double bonds, triple bonds or combinations thereof. Examples of unsaturated alkyl groups include but are not limited to, vinyl, propenyl, crotyl, 2-isopentenyl, allenyl, butenyl, butadienyl, pentenyl, pentadienyl, 3-(1,4-pentadienyl), hexenyl, hexadienyl, ethynyl, propynyl, butynyl, and higher homologs and isomers. The term “C₁₋ₘ-alkyl” refers to an alkyl having from 1 to about m carbon atoms. The alkyl group may be optionally substituted with one or more substituents, 1 to 5 substituents, at any available point of attachment, as defined below.

The term “aryl”, unless otherwise specifically defined, refers to cyclic, aromatic hydrocarbon groups that have 1 to 5 aromatic rings, including monocyclic or bicyclic groups such as phenyl, biphenyl or naphthyl. Where containing two or more aromatic rings (bicyclic, etc.), the aromatic rings of the aryl group may be joined at a single point (e.g., biphenyl), or fused (e.g., naphthyl, phenanthrenyl and the like). The aryl group may be optionally substituted by one or more substituents, e.g., 1 to 5 substituents, at any point of attachment. In addition to the substituents described under the definition of “substituted,” other exemplary substituents include, but are not limited to, nitro, cycloalkyl or substituted cycloalkyl, cycloalkenyl or substituted cycloalkenyl, cyano, alkyl, fused cyclic groups, fused cycloalkyl, fused cycloalkenyl, fused heterocycle, and fused aryl, and those groups recited above as exemplary alkyl substituents. The substituents can themselves be optionally substituted.

The term “heteroaryl”, unless otherwise specifically defined refers to cyclic, aromatic hydrocarbon groups that have 1 to 5 aromatic rings, including monocyclic or bicyclic groups, which contain at least one heteroatom such as N, S, or O, such as pyridine, or quinoline. Where containing two or more aromatic rings (bicyclic, etc.), the aromatic rings of the aryl group may be joined at a single point (e.g., phenyl-pyridine), or fused (e.g., quinoline and the like). The aryl group may be optionally substituted by one or more substituents, e.g., 1 to 5 substituents, at any point of attachment. In addition to the substituents described under the definition of “substituted,” other exemplary substituents include, but are not limited to, nitro, cycloalkyl or substituted cycloalkyl, cycloalkenyl or substituted cycloalkenyl, cyano, alkyl, fused cyclic groups, fused cycloalkyl, fused cycloalkenyl, fused heterocycle, and fused aryl, and those groups recited above as exemplary alkyl substituents. The substituents can themselves be optionally substituted.

The terms “heterocycle” and “heterocyclic”, unless otherwise specifically defined, refer to fully saturated, or partially or fully unsaturated, including aromatic (i.e., “heteroaryl”) cyclic groups (for example, 4 to 7 membered monocyclic, 7 to 12 membered bicyclic, or 8 to 16 membered tricyclic ring systems) which have at least one heteroatom in at least one carbon atom-containing ring. Each ring of the heterocyclic group containing a heteroatom may have 1, 2, 3, or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached to the remainder of the molecule at any heteroatom or carbon atom of the ring or ring system. Exemplary monocyclic heterocyclic groups include, but are not limited to, azetidinyl, pyrrolidinyl, pyrrolyl, pyrazolyl, oxetanyl, dioxanyl, dioxolanyl, oxathiolanyl, pyrazolinyl, imidazolyl, imidazolinyl, imidazolidinyl, oxazolyl, oxazolidinyl, isoxazolinyl, isoxazolyl, thietanyl, azetidine, diazetidine, thiolanyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolidinyl, 2-oxoazepinyl, azepinyl, hexahydrodiazepinyl, 4-piperidonyl, pyridyl, purinyl, pyrazinyl, pyrimidinyl, pyridazinyl, triazinyl, triazolyl, tetrazolyl, tetrahydropyranyl, morpholinyl, thiamorpholinyl, thiamorpholinyl sulfoxide, thiamorpholinyl sulfone, 1,3-dioxolane and tetrahydro-1,1-dioxothienyl, and the like. Exemplary bicyclic heterocyclic groups include, but are not limited to, indolyl, isoindolyl, benzothiazolyl, benzoxazolyl, benzoxadiazolyl, benzothienyl, benzo[d][1,3]dioxolyl, 2,3-dihydrobenzo[b][1,4]dioxinyl, quinuclidinyl, quinolinyl, tetrahydroisoquinolinyl, isoquinolinyl, benzimidazolyl, benzopyranyl, indolizinyl, benzofuryl, benzofurazanyl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl] or furo[2,3-b]pyridinyl), dihydroisoindolyl, dihydroquinazolinyl (such as 3,4-dihydro-4-oxo-quinazolinyl), triazinylazepinyl, tetrahydroquinolinyl and the like. Exemplary tricyclic heterocyclic groups include, but are not limited to, carbazolyl, benzidolyl, phenanthrolinyl, acridinyl, phenanthridinyl, xanthenyl, and the like.

A heterocyclic group may be optionally “substituted” with one or more substituents, 1 to 5 substituents, at any available point of attachment. In addition to the substituents described under the definition of “substituted,” other exemplary substituents include, but are not limited to, cycloalkyl or substituted cycloalkyl, cycloalkenyl or substituted cycloalkenyl, nitro, oxo (i.e., ═O), cyano, alkyl or substituted alkyl, spiro-attached or fused cyclic substituents at any available point or points of attachment, spiro-attached cycloalkyl, spiro-attached cycloalkenyl, Spiro-attached heterocycle (excluding heteroaryl), fused cycloalkyl, fused cycloalkenyl, fused heterocycle, fused aryl, and the like. The substituents can themselves be optionally substituted.

The term “substituted” means substituted by a below-described substituent group in any possible position. Substituent groups for the above moieties useful in this disclosure are those groups that do not significantly diminish the biological activity of the disclosed compound. Substituent groups that do not significantly diminish the biological activity of the disclosed compound include, but are not limited to, H, halogen, N₃, NCS, CN, NO₂, NX₁X₂, OX₃, C(X₃)₃, OAc, O-acyl, O-aroyl, NH-acyl, NH-aroyl, NHCOalkyl, CHO, C(halogen)₃, Ph, OPh, CH₂Ph, OCH₂Ph, COOX₃, SO₃H, PO₃H₂, SO₂NX₂X₂, CONX₁X₂, alkyl, alcohol, alkoxy, dioxolanyl, alkylmercapto, dithiolanyl, dithianyl, alkylamino, dialkylamino, sulfonamide, thioalkoxy or methylene dioxy when the substituted structure has two adjacent carbon atoms, wherein X₁ and X₂ each independently comprise H or alkyl, and X₃ comprises H, alkyl, hydroxy lower alkyl. Unless otherwise specifically limited, a substituent group may be in any possible position.

The term “prodrug,” as used in this disclosure, means a compound which is convertible in vivo by metabolic means (e.g., by hydrolysis) to a compound of Formula (I),

The terms “salt” or “salts”, as employed in this disclosure, denote acidic and/or basic salts formed with inorganic and/or organic acids and bases.

The term “tautomer” as used in this disclosure refers to compounds produced by the phenomenon wherein a proton of one atom of a molecule shifts to another atom. (March, Advanced Organic Chemistry: Reactions, Mechanisms and Structures, 4th Ed., John Wiley & Sons, pp. 69-74 (1992)).

The following abbreviations are used in this disclosure and have the following definitions: DMF is dimethylformamide; DMSO is dimethylsulfoxide; THF is tetrahydrofuran; and Tris is tris(hydroxymethyl)aminomethane.

The term “carrier”, as used in this disclosure, encompasses carriers, excipients, and diluents and means a material, composition or vehicle, such as a liquid or solid filler, diluent, excipient, solvent or encapsulating material, involved in carrying or transporting a pharmaceutical agent from one organ, or portion of the body, to another organ, or portion of the body.

The phrase “pharmaceutically acceptable” is employed in this disclosure to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “administer”, “administering”, or “administration” as used in this disclosure refer to either directly administering a compound or pharmaceutically acceptable salt of the compound or a composition to a subject, or administering a prodrug derivative or analog of the compound or pharmaceutically acceptable salt of the compound or composition to the subject, which can form an equivalent amount of active compound within the subject's body.

As used herein, a “potentiator” or a “compound that potentiates” is a compound that supplements or enhances the activity of an antifungal agent, e.g., the antifungal activity of an antifungal agent. In some embodiments, the potentiator is not an antifungal agent, i.e., does not exhibit antifungal activity on its own. In other embodiments, the potentiator is an antifungal agent itself. In some embodiments, the activity of the antifungal agent is synergistic with the activity of the potentiator,

The term “enhances”, as used herein, means augments, increases, intensifies, makes greater, improves, and/or acts synergistically with. For example, a first compound that enhances the activity of a second compound augments, increases, intensifies, makes greater, improves the activity of, and/or acts synergistically with, the second compound.

An “effective amount”, when used in connection with a composition described herein, is an amount effective for treating a fungal infection, or for inhibiting the growth of, or killing, a fungus.

A “subject”, as used herein, is a mammal, e.g., a human, mouse, rat, guinea pig, dog, cat, horse, cow, pig, or a non-human primate, such as a monkey, chimpanzee, baboon, or rhesus.

As used herein, “treat”, “treating” or “treatment” refers to administering a therapy in an amount, manner (e.g., schedule of administration), and/or mode (e.g., route of administration), effective to improve a disorder (e.g., an infection described herein) or a symptom thereof, or to prevent or slow the progression of a disorder (e.g., an infection described herein) or a symptom thereof. This can be evidenced by, e.g., an improvement in a parameter associated with a disorder or a symptom thereof, e.g., to a statistically significant degree or to a degree detectable to one skilled in the art. An effective amount, manner, or mode can vary depending on the subject and may be tailored to the subject. By preventing or slowing progression of a disorder or a symptom thereof, a treatment can prevent or slow deterioration resulting from a disorder or a symptom thereof in an affected or diagnosed subject.

As used herein, “administered in combination” means that two or more agents are administered to a subject at the same time or within an interval, such that there is overlap of an effect of each agent on the subject. The administrations of the first and second agent can be spaced sufficiently close together such that a combinatorial effect, e.g., a synergistic effect, is achieved. The interval can be an interval of hours, days or weeks. The agents can be concurrently bioavailable, e.g., detectable, in the subject. For example, at least one administration of one of the agents, e.g., an antifungal agent, can be made while the other agent, e.g., a compound described herein, is still present at a therapeutic level in the subject. The subject may have had a response that did not meet a predetermined threshold. For example, the subject may have had a failed or incomplete response, e.g., a failed or incomplete clinical response to the antifungal agent. An antifungal agent and a compound described herein may be formulated for separate administration or may be formulated for administration together.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### Methods of Identifying Potentiators

In some instances, the methods described herein are useful for identifying compounds that potentiate the activity of an antifungal agent. The rationale is to screen compounds using fungal strains that are treated with an antifungal agent. The screening methods are readily adapted to high throughput screening (HTS).

In one nonlimiting example, the screen involves contacting a fungus with an antifungal agent. The screen further involves contacting the fungus with a candidate compound. The screen also involves comparing the number of viable cells of the fungus in the presence of the candidate compound to the number of viable cells of the fungus in the absence of the candidate compound. A greater number of viable cells in the absence of the candidate compound compared to the number of viable cells in the presence of the candidate compound is indicative that the candidate compound is a potentiator.

In some situations, the method further includes contacting a second fungus with the candidate compound in the absence of the antifungal agent, and determining the number of viable cells of the second fungus in the absence and presence of the candidate compound, wherein the fungus and the second fungus are the same.

The number of viable cells can be determined by any method known in the art. For example, the fungal cells can be visualized with dyes that discriminate between living and dead cells. Exemplary dyes are alamar blue, XTT, fluorescein diacetate, and those in the LIVE/DEAD® Yeast Viability Kit (Invitrogen). Other nonlimiting examples are described in U.S. Pat. Nos. 5,445,946 and 5,437,980; and in Jin et al., Mycopathol. 159:353-360 (2005).

In some instances, the assay is performed on cells grown in a liquid growth medium. In other instances, the number of viable cells is determined in a plate assay, e.g., using cells grown on a microtiter plate.

The screening method can be conducted on any fungus, e.g., one or more of the following: a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

The potentiators identified in the screens can be used to inhibit, reduce, prevent growth of and/or kill a fungus. Such a fungus can be wherever the fungus grows, including within a subject, such as a mammal. Thus, the potentiators can be used to treat a fungal infection in a subject.

In the screens described herein, any candidate compound can be assayed to determine if it has potentiating capacity. For example, a candidate compound library can be used to provide a candidate compound. Nonlimiting examples of candidate compound libraries include The Compound Library of the New England Regional Center of Excellence for Biodefense and Emergine Infectious Diseases, The Compound Library of the National Institutes of Health Molecular Library Screening Center, The ChemBridge Library, the ChemDiv Library, and the MayBridge Library. Alternatively, a candidate compound can be synthesized using known methods.

### The Compounds of Formula I

The compositions and methods described herein include compounds according to Formula I:

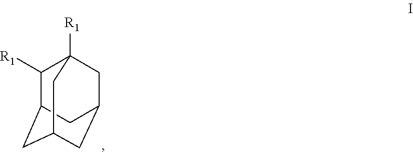

or pharmaceutically acceptable salts, hydrates, solvates, or prodrugs thereof:

wherein R₁ is as described above for Formula I.

Nonlimiting illustrative compounds of Formula I include:

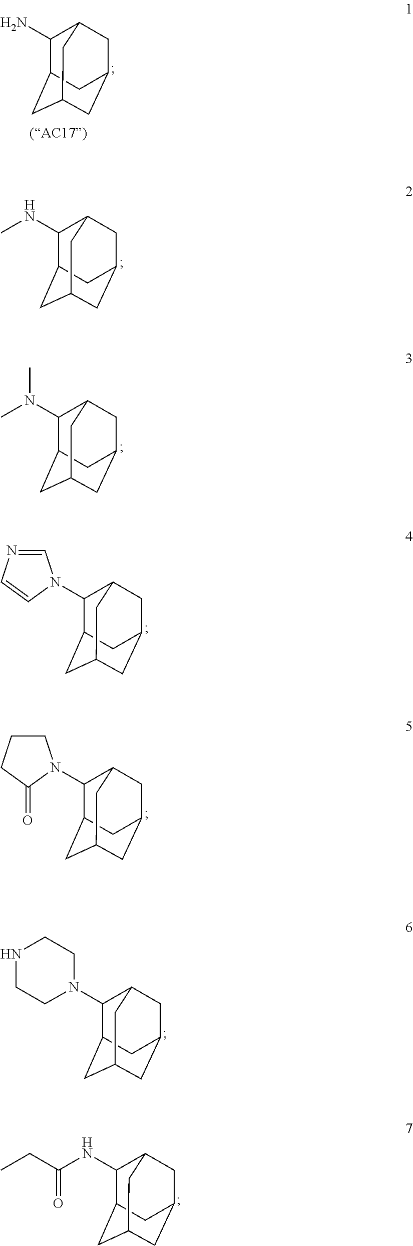

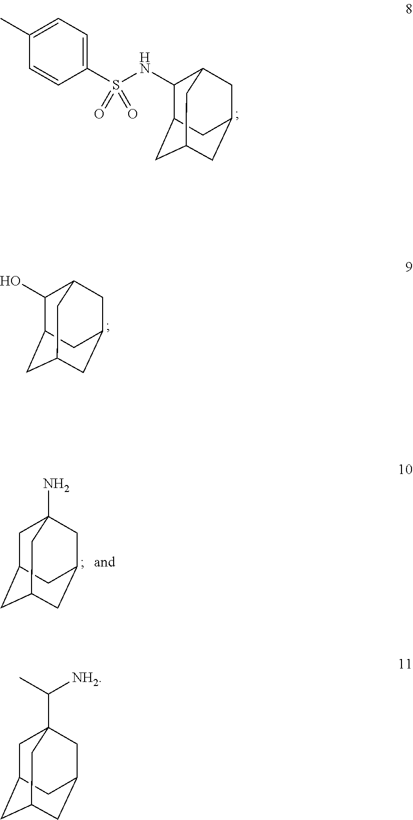

The compositions and methods described herein include compounds according to Formula Ia:

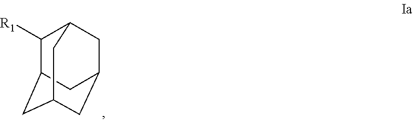

or pharmaceutically acceptable salts, hydrates, solvates, or prodrugs thereof:

wherein R₁ is as described above for Formula Ia. Nonlimiting illustrative compounds of Formula Ia include:

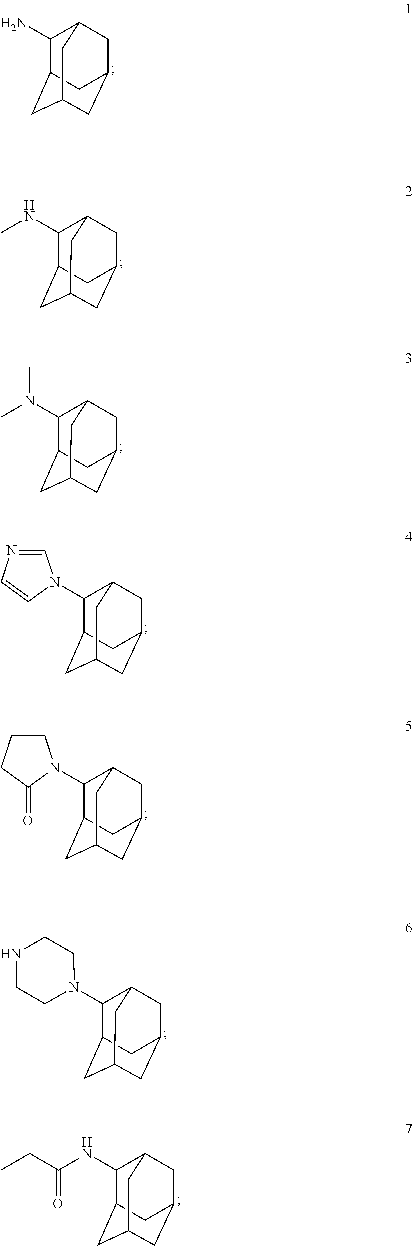

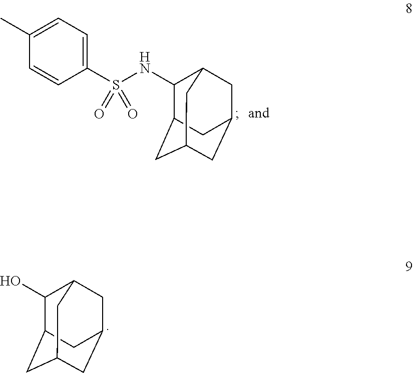

The compositions and methods described herein include compounds according to Formula Ib:

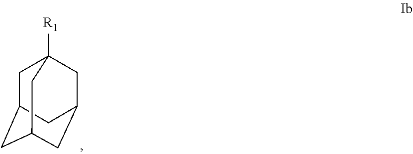

or pharmaceutically acceptable salts, hydrates, solvates, or prodrugs thereof:

wherein R₁ is as described above for Formula Ib. Nonlimiting illustrative compounds of Formula Ib include:

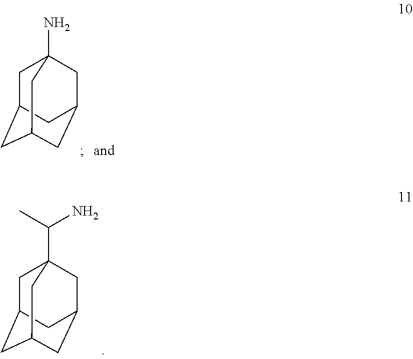

The compounds of Formula I can also form salts which are also within the scope of this disclosure. Reference to a compound of the present disclosure is understood to include reference to salts thereof, unless otherwise indicated. The compounds of Formula I may form pharmaceutically acceptable (i.e., non-toxic, physiologically-acceptable) salts as well as other salts that are also useful, e.g., in isolation or purification steps which can be employed during preparation.

The compounds of Formula I which contain a basic moiety, such as, but not limited to, an amine or a pyridine or imidazole ring, can form salts with a variety of organic and inorganic acids. Exemplary acid addition salts include, but are not limited to, acetates (such as those formed with acetic acid or trihaloacetic acid, e.g., trifluoroacetic acid), adipates, alginates, ascorbates, aspartates, benzoates, benzenesulfonates, bisulfates, borates, butyrates, citrates, camphorates, camphorsulfonates, cyclopentancpropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, hydroxyethanesulfonates (e.g., 2-hydroxyethanesulfonates), lactates, maleates, methanesulfonates, naphthalenesulfonates (e.g., 2-naphthalenesulfonates), nicotinates, nitrates, oxalates, pectinates, persulfates, phenylpropionates (e.g., 3-phenylpropionates), phosphates, picrates, pivalates, propionates, salicylates, succinates, sulfates (such as those formed with sulfuric acid), sulfonates, tartrates, thiocyanates, toluenesulfonates such as tosylates, undecanoates, and the like.

The compounds of Formula I which contain an acidic moiety, such as, but not limited to, a carboxylic acid, can form salts with a variety of organic and inorganic bases. Exemplary basic salts include, but are not limited to, ammonium salts, alkali metal salts such as sodium, lithium and potassium salts, alkaline earth metal salts such as calcium and magnesium salts, salts with organic bases (e.g., organic amines) such as benzathines, dicyclohexylamines, hydrabamines (formed with N,N-bis(dehydroabietyl)ethylenediamine), N-methyl-D-glucamines, N-methyl-D-glycamides, t-butyl amines, and salts with amino acids such as arginine, lysine and the like. Basic nitrogen-containing groups can be quaternized with agents such as lower alkyl halides (e.g., methyl, ethyl, propyl, and butyl chlorides, bromides and iodides), dialkyl sulfates (e.g., dimethyl, diethyl, dibutyl, and diamyl sulfates), long chain halides (e.g., decyl, lauryl, myristyl and stearyl chlorides, bromides and iodides), aralkyl halides (e.g., benzyl and phenethyl bromides), and the like.

Exemplary nonlimiting compounds of Formula I are listed in the Examples section below. Solvates of the compounds of this disclosure, including hydrates of the compounds, as well as mixtures of the hydrate- and the keto-form of the compounds, are within the scope of this disclosure.

### Methods of Making Adamantane Derivatives

The compounds described herein can be synthesized by chemical means as described in the following generic schemes and in the Examples below. The compounds may be synthesized from commercially available starting material and need not be made exclusively by the illustrative syntheses. A person of skill in the art understands that additional methods of making the compounds exist. A person of skill in the art also understands that additional general synthetic schemes for the compounds disclosed herein can be understood from the illustrative schemes below. Alternatively, all of the compounds disclosed herein are, at the time of tiling, commercially available (e.g., from Ambinter, Paris France; Sigma Aldrich, St. Louis, Mo.; Ryan Scientific, Mt. Pleasant S.C.; Enamine, Kiev, Ukraine, ASDI Biosciences, Newark Del.).

Scheme 1 shows the direct transformation of adamantane to the hydroxyl substituted adamantane.

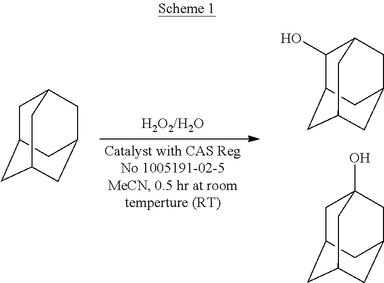

Both hydroxy substituted adamantanes can be produced according to the chemistry shown in Scheme 1 and as described in Alonso et al., Tetrahedron, 64 (8), 1847-1852 (2008).

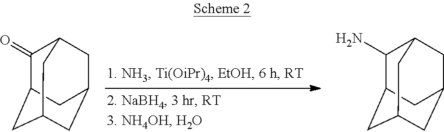

The aminoadamantane of Scheme 2 (“AC17”) can be synthesized according to the method described in Miriyala et al., Tetrahedron, 60(7), 1463-1472, (2004).

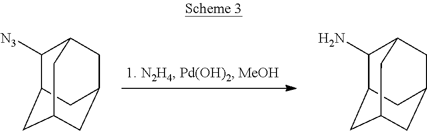

Alternatively, the aminoadamantane of Scheme 2 can be produced by the method described in Malik et al., Synthesis, 6:450-451, (1989).

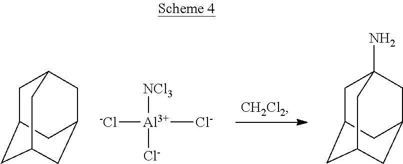

Amantidine, the aminoadamantane derivitive in Scheme 5 can be synthesized by the methods described in Wipf, Encyclopedia of Reagents for Organic Synthesis. Ed., Peter Wipf, Wiley and Sons: Chichester, 2005.

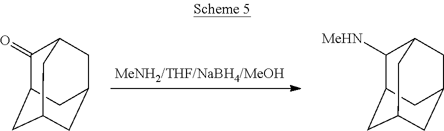

The methylamino derivative of adamantine can be synthesized using the reagents listed in Scheme 5, as described in Jones et al., J. Org. Chem., 63 (8), 2758-2760 (1998).

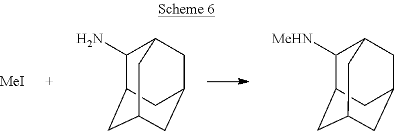

Alternatively, the methylamino derivative of adamantine can be synthesized using the reagents listed in Scheme 6 as described in U.S. Pat. No. 4,826,667.

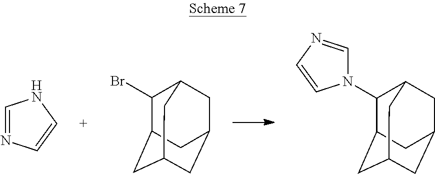

The imidazole derivative of adamantane can be synthesized listed in Scheme 7 as described in Matolcsy et al., Acta Phytopathol. Acad. Sci. Hung., 13 (1-2), 223-225 (1978).

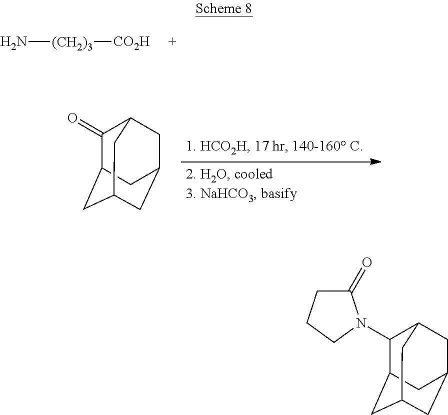

Alternatively, the pyrrolo derivative of adamantine can be synthesized using the reagents listed in Scheme 8 as described in PCT International Appl. 2005/108361.

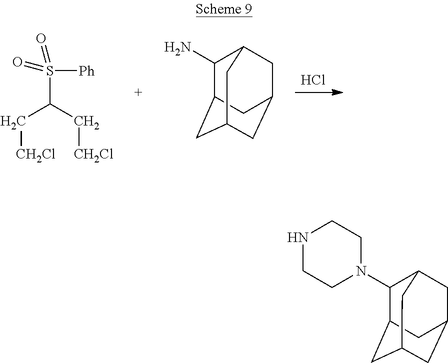

The piperazine derivative of adamantane can be synthesized according to the method described in Klimova et al., Khimiko-Farmatsevticheskii Zhurnal, 9(11), 8-11 (1975).

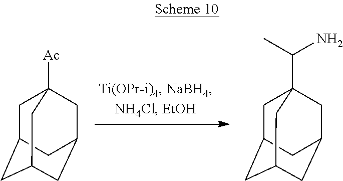

Rimantadine can be synthesized according to the method described in Bhattacharyya, J. Chem. Soc., Perkin Trans. 1: Organic and Bio-Organic Chemistry, 14, 1845-1847 (1995).

### Methods of Treating Fungal Infections

Some potentiator compounds described herein can be used in combination with known antifungal agents to treat a variety of fungal infections, but have no antifungal activity of their own. Additionally, certain potentiator compounds have antifungal activity, but also act to potentiate the activity of an antifungal agent as well.

Fungi and Fungal Infections

Fungal infections are caused by a number of fungal species, and the compounds described herein can be used to inhibit the growth of, or kill, such fungal species. These fungi include, but are not limited to, a member of the genus Aspergillus (e.g., Aspergillus flavus, Aspergillus fumigatus, Aspergillus glaucus, Aspergillus nidulans, Aspergillus niger, and Aspergillus terreus); Blastomyces dermatitidis; a member of the genus Candida (e.g., Candida albicans, Candida glabrata, Candida tropicalis, Candida parapsilosis, Candida krusei, and Candida guillermondii); Coccidioides immitis; a member of the genus Cryptococcus (e.g., Cryptococcus neoformans, Cryptococcus albidus, and Cryptococcus laurentii); Histoplasma capsulatum var. capsulatum; Histoplasma capsulatum var. duboisii; Paracoccidioides brasiliensis; Sporothrix schenckii; Absidia corymbifera; Rhizomucor pusillus; and Rhizopus arrhizus.

These fungal species mediate a number of fungal infections including, but not limited to, aspergillosis, blastomycosis, candidiasis (e.g., oral thrush or vaginosis), coccidioidomycosis, cryptococcosis, histoplasmosis, paracoccidiomycosis, sporotrichosis, and zygomycosis. Any of these fungal infections can be treated with the compounds and methods described herein. In certain instances, the fungal infection is an infection mediated by Candida albicans, such as oral candidiasis (Thein et al., Arch. Oral Biol. 52:1200-1208 (2007)) or vaginitis (Rex et al., Clin. Infect. Dis. 30:662-678 (2000)).

Some fungal infections can be associated with indwelling devices, such as catheters and prostheses. For example, fungal biofilms are a major problem in catheters and other device-related infections (Kuhn et al., Curr. Opin. Investig. Drugs 5:186-197 (2004); Ramage et al., FEMS Yeast Res. 6:979-986 (2006)), and the compounds described herein can be used to treat them. For example, to treat a Candida albicans infection of a medical device, the compounds described herein can be applied to its surface as a coating. In other instances, e.g., for dentures or catheters, the compounds described herein can be applied directly to the device (Nikawa et al., Int. J. Prosthodont. 8:434-444 (1995); Sherertz et al., Antimicrob. Agents Chemother. 50:1865-1868 (2006); Fortun, Enferm. Infecc. Microbiol. Clin. 26:168-174 (2008)).

In some situations, the compounds described herein can also be used to treat such infections and diseases in immunodeficient subjects, such as neutropenic subjects undergoing chemotherapy. In other instances, the subject can be undergoing or have undergone an additional therapy, e.g., antibiotic therapy.

Antifungal Agents

The potentiator compounds described herein can be used in combination with any known antifungal agent. Useful antifungal agents include, but are not limited to, Amphotericin (e.g., Amphotericin B, Amphotericin B Lipid Complex (ABLC), Liposomal Amphotericin B (L-AMB), and Amphotericin B Colloidal Dispersion (ABCD)), azoles (e.g., an imidazole (e.g., miconazole, e.g., Monistat®), clotrimazole, fluconazole, itraconazole, ketoconazole, ravuconazole, posaconazole, and voriconazole), caspofungin, micafungin, FK463, anidulafungin (LY303366), hydroxystilbamidine, 5-fluorocytosine, flucytosine, iodide (e.g., as a saturated solution of potassium iodide, or SSKI), terbinafine, Nystatin, griseofulvin, and ciclopirox. One exemplary antifungal agent is miconazole, e.g., Monistat®, which is an imidazole antifungal agent commonly applied topically to treat fungal infections. These and other antifungal agents are known to those of ordinary skill in the art and available commercially. For example, many of these antifungal agents are commercially available from Pfizer Inc.; McNeil-PPC, Inc; Johnson & Johnson; Enzon Pharmaceuticals, Inc.; Schering-Plough HealthCare Products; Sandoz Inc.: Ranbaxy Laboratories Ltd.; Mylan Pharmaceuticals, Inc.; Roxane Laboratories, Inc.; Sicor Pharmaceuticals, Inc.; Novopharm Ltd.; Apotex Inc.; Bedford Laboratories; Pliva Inc.; Taro Pharmaceutical Industries, Ltd.; and American Pharmaceutical Partners, Inc.

Therapeutic Administration

The route and/or mode of administration of an antifungal agent and a potentiator compound described herein can vary depending upon the desired results. For example, the doses of the antifungal agent and a compound described herein can be chosen such that the therapeutic effect is at least 10%, 20%, 25%, 30%, 40%, 50%, 60%, 70%, 80%, 90%, 100%, 125%, 150%, 175%, or 200% greater than that achieved with the antifungal agent alone (i.e., in the absence of a compound described herein). Such effects can be recognized by those skilled in the art, e.g., using standard parameters associated with fungal infections. Dosage regimens can be adjusted to provide the desired response, e.g., a therapeutic response or a combinatorial therapeutic effect. Generally, any combination of doses (either separate or co-formulated) of an antifungal agent and a compound described herein can be used in order to provide a subject with both agents in bioavailable quantities.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration can result in release of a potentiator and/or an antifungal agent described herein into the bloodstream. The mode of administration is left to the discretion of the practitioner.

In some instances, a potentiator and/or an antifungal agent described herein can be administered locally. This can be achieved, for example, by local infusion during surgery, topical application (e.g., in a cream or lotion), by injection, by means of a catheter, by means of a suppository or enema, or by means of an implant, said implant being of a porous, non-porous, or gelatinous material, including membranes, such as sialastic membranes, or fibers.

In some situations, a potentiator and/or an antifungal agent described herein can be introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

This disclosure also features a device for administering an anti fungal agent and a compound described herein. The device can include, e.g., one or more housings for storing pharmaceutical compositions, and can be configured to deliver unit doses of an antifungal agent and a compound described herein. The antifungal agent and a compound described herein can be stored in the same or separate compartments. For example, the device can combine the antifungal agent and the compound prior to administration. It is also possible to use different devices to administer the antifungal agent and a compound described herein.

Pulmonary administration can also be employed, e.g., by use of an inhaler or nebulizer, and formulation with an aerosolizing agent, or via perfusion in a fluorocarbon or synthetic pulmonary surfactant.

In some instances, a potentiator and/or an antifungal agent described herein can be delivered in a vesicle, in particular a liposome (see Langer, Science 249:527-1533 (1990); and Treat et al., Liposomes in the Therapy of Infectious Disease and Cancer, Gabriel Lopez-Berestein, John Wiley & Sons Canada, pp. 317-327 and pp. 353-365 (1989)).

In yet other situations, a potentiator and/or an antifungal agent described herein can be delivered in a controlled-release system or sustained-release system (see, e.g., Goodson, in Medical Applications of Controlled Release, Robert L. Langer, Donald Lee Wise, CRC Press, 2:115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref Biomed Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise, eds., 1974, CRC Press); Controlled Drug Bioavailability, Drug Product Design and Performance (Smolen and Ball eds., John Wiley, 1984); Ranger et al., J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet other situations, a controlled- or sustained-release system can be placed in proximity of a target of a potentiator and/or an antifungal agent described herein, e.g., the reproductive organs, reducing the dose to a fraction of the systemic dose.

A potentiator and/or an antifungal agent described herein can be formulated as a pharmaceutical composition that includes a suitable amount of a physiologically acceptable excipient (see, e.g., Remington's Pharmaceutical Sciences pp. 1447-1676 (Alfonso R. Gennaro, ed., 19th ed. 1995)). Such physiologically acceptable excipients can be, e.g., liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an animal. The physiologically acceptable excipient should be stable under the conditions of manufacture and storage and should be preserved against the contaminating action of microorganisms. Water is a particularly useful excipient when a potentiator and/or an antifungal agent described herein is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Other examples of suitable physiologically acceptable excipients are described in Remington's Pharmaceutical Sciences pp. 1447-1676 (Alfonso R. Gennaro, ed., 19th ed. 1995). The pharmaceutical compositions, if desired, can also contain minor amounts of wetting or emulsifying agents, or pH buffering agents.

Liquid carriers can be used in preparing solutions, suspensions, emulsions, syrups, and elixirs. A potentiator and/or an antifungal agent described herein can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives described herein, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. The liquid carriers can be in sterile liquid form for administration. The liquid carrier for pressurized compositions can be halogenated hydrocarbon or other pharmaceutically acceptable propellant.

A potentiator and/or an antifungal agent described herein can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule.

In some instances, a potentiator and/or an antifungal agent described herein is formulated in accordance with routine procedures as a composition adapted for oral administration to humans. Compositions for oral delivery can be in the form of e.g., tablets, lozenges, buccal forms, troches, aqueous or oily suspensions or solutions, granules, powders, emulsions, capsules, syrups, or elixirs. Orally administered compositions can contain one or more additional agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with a finely divided antifungal agent and/or compound described herein. In tablets, a potentiator and/or an antifungal agent described herein can be mixed with a carrier having compression properties in suitable proportions and compacted in the shape and size desired. The powders and tablets can contain up to about 99% of a potentiator and/or an antifungal agent described herein.

Capsules can contain mixtures of a potentiator and/or an antifungal agent described herein with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cctostearl alcohol, cctomacrogol emulsifying wax, sorbitan esters, colloidal silicon dioxide, phosphates, sodium dodccylsulfate, magnesium aluminum silicate, and triethanolamine.

Moreover, when in a tablet or pill form, a potentiator and/or an antifungal agent described herein can be coated to delay disintegration and absorption in the gastrointestinal tract, thereby providing a sustained action over an extended period of time. Selectively permeable membranes surrounding an osmotically active driving a potentiator and/or an antifungal agent described herein can also be suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In some situations, the excipients are of pharmaceutical grade.

In other instances, a potentiator and/or an antifungal agent described herein can be formulated for intravenous administration. Compositions for intravenous administration can comprise a sterile isotonic aqueous buffer. The compositions can also include a solubilizing agent. Compositions for intravenous administration can optionally include a local anesthetic such as lignocaine to lessen pain at the site of the injection. The ingredients can be supplied either separately or mixed together in unit dosage form, for example, as a dry lyophilized powder or water-free concentrate in a hermetically sealed container such as an ampule or sachette indicating the quantity of active agent. Where a potentiator and/or an antifungal agent described herein is administered by infusion, it can be dispensed, for example, with an infusion bottle containing sterile pharmaceutical grade water or saline. Where a potentiator and/or an antifungal agent described herein is administered by injection, an ampule of sterile water for injection or saline can be provided so that the ingredients can be mixed prior to administration.

In other circumstances, a potentiator and/or an antifungal agent described herein can be administered across the surface of the body and the inner linings of the bodily passages, including epithelial and mucosal tissues. Such administrations can be carried out using a potentiator and/or an antifungal agent described herein in lotions, creams, foams, patches, suspensions, solutions, and suppositories (e.g., rectal or vaginal). In some instances, a transdermal patch can be used that contains a potentiator and/or an antifungal agent described herein and a carrier that is inert to the antifungal agent and/or compound described herein, is non-toxic to the skin, and that allows delivery of the agent for systemic absorption into the blood stream via the skin. The carrier can take any number of forms such as creams or ointments, pastes, gels, or occlusive devices. The creams or ointments can be viscous liquid or semisolid emulsions of either the oil-in-water or water-in-oil type. Pastes of absorptive powders dispersed in petroleum or hydrophilic petroleum containing a potentiator and/or an antifungal agent described herein can also be used. A variety of occlusive devices can be used to release a potentiator and/or an antifungal agent described herein into the blood stream, such as a semi-permeable membrane covering a reservoir containing the antifungal agent and/or compound described herein with or without a carrier, or a matrix containing the antifungal agent and/or compound described herein.

A potentiator and/or an antifungal agent described herein can be administered rectally or vaginally in the form of a conventional suppository. Suppository formulations can be made using methods known to those in the art from traditional materials, including cocoa butter, with or without the addition of waxes to alter the suppository's melting point, and glycerin. Water-soluble suppository bases, such as polyethylene glycols of various molecular weights, can also be used.

The amount of a potentiator and/or an antifungal agent described herein that is effective for treating an infection can be determined using standard clinical techniques known to those will skill in the art. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. For example, the dose of a potentiator and/or an antifungal agent described herein can each range from about 0.001 mg/kg to about 250 mg/kg of body weight per day, from about 1 mg/kg to about 250 mg/kg body weight per day, from about 1 mg/kg to about 50 mg/kg body weight per day, or from about 1 mg/kg to about 20 mg/kg of body weight per day. Equivalent dosages can be administered over various time periods including, but not limited to, about every 2 hrs, about every 6 hrs, about every 8 hrs, about every 12 hrs, about every 24 hrs, about every 36 hrs, about every 48 hrs. about every 72 hrs, about every week, about every two weeks, about every three weeks, about every month, and about every two months. The number and frequency of dosages corresponding to a completed course of therapy can be determined according to the judgment of a health-care practitioner.

In some instances, a pharmaceutical composition described herein is in unit dosage form, e.g., as a tablet, capsule, powder, solution, suspension, emulsion, granule, or suppository. In such form, the pharmaceutical composition can be sub-divided into unit doses containing appropriate quantities of a potentiator and/or an antifungal agent described herein. The unit dosage form can be a packaged pharmaceutical composition, for example, packeted powders, vials, ampoules, pre-filled syringes or sachets containing liquids. The unit dosage foil can be, for example, a capsule or tablet itself, or it can be the appropriate number of any such compositions in package form. Such unit dosage form can contain from about 1 mg/kg to about 250 mg/kg, and can be given in a single dose or in two or more divided doses.

The invention is further described in the following examples, which do not limit the scope of the invention described in the claims.

### EXAMPLES

### Example 1

### Characterization of C. albicans Persisters

Both planktonic and biofilm populations were examined for the possible presence of persisters. Several compounds including amphotericin B, chlorhexidine, and caspofungin kill Candida biofilms, and these were tested in dose-dependent experiments. A biphasic killing curve revealing a subpopulation of survivors indicates the presence of persister cells.

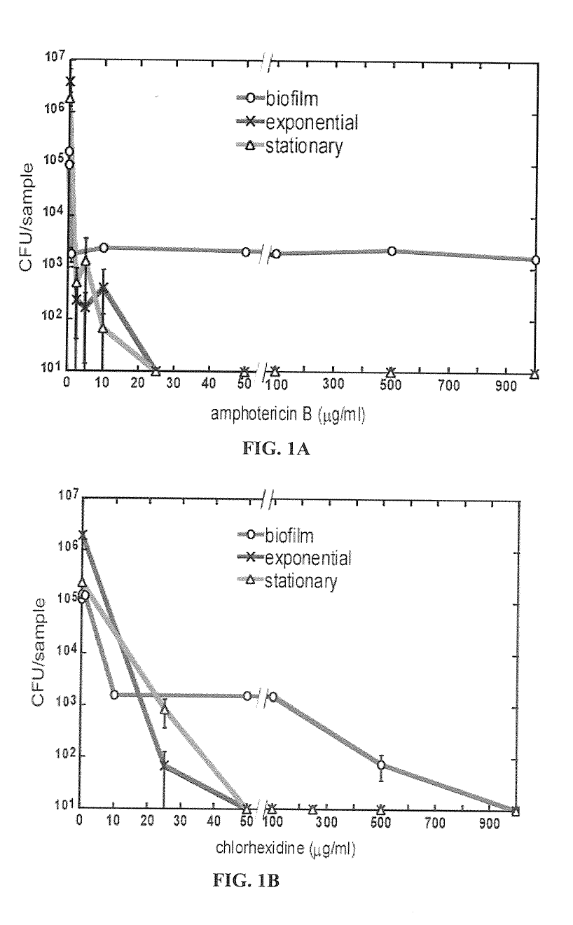

Biofilms of C. albicans 3153A cells were cultured in wells of microtiter plates in RPMI medium for 48 hrs (Ramage et al., Antimicrob. Agents Chemother. 45:2475-2479 (2001)), washed twice in PBS, pH 7.4, to remove nonadherent cells, and resuspended in 100 μl RPMI growth medium containing antifungals. After 24 hrs of antifungal challenge, the biofilms and cultures were washed twice, resuspended in 100 μl PBS, scraped, transferred into eppendorf tubes, vortexed and plated for colony forming unit (CFU) determination on YPD medium. Microscopy indicated that the material was a mixture of single cells and clumps of ≦10 cells. This could lead to an underestimation of surviving cells by a factor of ≦10. In parallel, exponentially growing and stationary planktonic cultures were grown for 48 hrs in RPMI medium, and then antifungals were added for 24 hrs. The experiment was performed in triplicate and error bars indicate standard deviation (see FIGS. 1A-1B).

Caspofungin had a limited effect on biofilms, producing ≦10 fold killing. Amphotericin B effectively killed exponentially growing and stationary cells, with little indication of surviving cells (FIG. 1A). By contrast, a biphasic killing was observed in Candida biofilms, with the majority of the population killed at low concentrations (but above the MIC of 1 μg/ml) while the remaining cells were unaffected by higher concentrations of the drug (FIG. 1A). More than 1% of cells appeared invulnerable to amphotericin B, indicating the presence of persisters in the yeast biofilm, in contrast to observations with bacteria, where stationary planktonic populations produce more persisters than the biofilm. Resistance to killing by amphotericin B, which makes “holes” in the membrane, was unexpected. The activity of this compound depends on, and is limited by, the availability of ergosterol.

Similar to the results seen with amphotericin B, chlorhexidine produced a biphasic killing of the biofilm, while cells in both exponential and stationary cultures were eliminated (FIG. 1B). At higher concentrations (above 100 μg/ml), killing of persisters was observed, and the biofilm was completely sterilized at 1000 μg/ml (a concentration 2-fold lower than what is commonly used in mouthwash and as a therapy for treatment of oral thrush caused by C. albicans (0.2%)).

The biphasic nature of the killing showed that resistant mutants were present in the population. In order to determine whether surviving cells were phenotypic variants of the wild type or whether they were mutants, resistance of the surviving cells was examined.

Biofilms were grown in microtiter plates and were treated with amphotericin B or chlorhexidine (100 μg/ml) for 24 hrs, after which they were washed and vortexed, as discussed above. The cells were then reinoculated into microtiter plates to form new biofilms. The new biofilms, derived from persisters that survived drug treatment, were again treated with the antifungal agents (as discussed above), and the procedure was repeated a total of 3 times. Biofilms were sampled for CFU determination before and after antifungal treatment. The experiment was performed in triplicate.

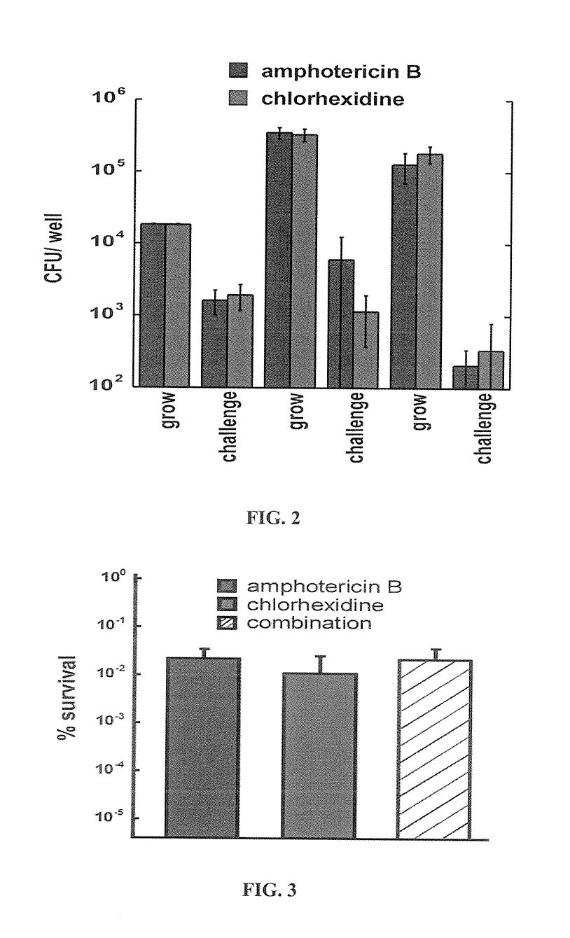

As demonstrated in FIG. 2, the population produced by surviving persisters was not more resistant to drugs, but rather gave rise to a new persister subpopulation (see FIG. 2; error bars indicate standard deviation). If the surviving cells were mutants, complete resistance would be expected upon reapplication of the antifungal or a progressive increase in the numbers of surviving cells with each treatment cycle. Thus, C. albicans persisters were phenotypic variants of the wild type that arose in a clonal population of genetically identical cells.

Tests were also performed to determine if yeast persisters were multidrug tolerant. Mature, 48 hr biofilms of C. albicans were challenged for 24 hrs with 100 μg/ml amphotericin B, 100 μg/ml chlorhexidine, or a combination of these two antifungal agents, using the same procedures discussed above. Biofilms were washed and sampled for CFU determination before and after antifungal treatment, as discussed above.

No additional killing was detected when biofilms were treated with both amphotericin B and chlorhexidine compared to cells treated with individual antifungal agents (FIG. 3; triplicate experiments with error bars indicating standard deviation). Similarly, the number of persisters was essentially the same (1%-3%) when biofilms were treated sequentially for 24 hrs with amphotericin B and then chlorhexidine, or vice versa. These experiments indicate the presence of a single uniform persister population.

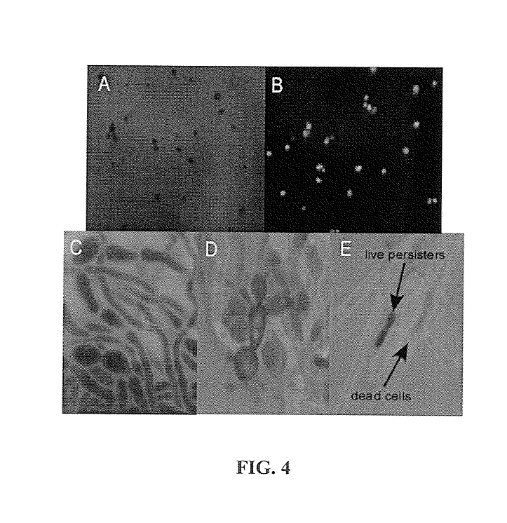

Persisters as in a biofilm were then visualized using several dyes, including fluorescein diacetate, which discriminate between live and dead fungal cells. Planktonic or biofilm cells were stained with 100 μg/ml fluorescein diacetate and examined by fluorescent microscopy. FIG. 4A depicts live planktonic cells; FIG. 4B depicts dead planktonic cells after treatment with 100 μg/ml amphotericin B (400× magnification); FIGS. 4C-4E, depict biofilms (1000× magnification) of untreated control, after 18 hrs or after 48 hrs of amphotericin B treatment (100 μg/ml), respectively.

Exponentially growing C. albicans cells killed with amphotericin B were readily stained with fluorescein diacetate as expected (FIGS. 4A-4B). A biofilm was then stained with fluorescein diacetate (FIGS. 4C-4E). After the addition of amphotericin B, there was a visible decrease in the number of live (dark) cells, and their morphology became aberrant (FIG. 4D). After 48 hrs of amphotericin B treatment, there were only a small number of unstained cells. They appeared as regular pseudohyphae or yeasts and were indistinguishable from morphologically normal untreated cells. Using fluorescence detection and forward scatter, dim persister cells were physically sorted from a disrupted biofilm and grown on agar medium. The sorted cells produced colonies on agar medium, confirming that they were alive. The ability to sort persisters is used to obtain their transcription profile using standard methods.

Given that persisters appeared only in the biofilm, their formation was dependent on the same genes/pathways that determine biofilm development. A large panel of biofilm-defective mutants was therefore tested for their ability to produce persisters by measuring survival to high levels of amphotericin B. These mutants were able to adhere to the surface of a microtiter plate, making it possible to assay in the biofilm survival protocol described above. All strains appeared to produce essentially normal levels of persisters (Table 1). This suggests that adherence, rather than subsequent biofilm formation, is the trigger for persister formation.

TABLE 1
Persister Formation by Biofilm-Deficient Strains of C. albicans.

| Strain | Genotype | Biofilm architecture | Persisters |
|---|---|---|---|
| 3153A | Control, wild type laboratory strain | Robust 3D wild type | +++¹ |
| CKY357 | CAI-4 mkc1Δ::hisG/mkc1Δ::hisG | Reduced filamentation | ++² |
| | mkc1::pCK70 (URA3) | | |
| CAI4 | SC5314 Δura3::λimm434/Δura3::λimm434 | Robust 3D wild type | +³ |
| CKY136 | CAI-4 efg1::hisG/efg1::hisG ade2::pDBI52 | Filamentation defect; | +++ |
| | (URA3) | sparse monolayer of cells | |
| CKY138 | CAI-4 efg1::hisG/efg1::hisG | Filamentation defect; | ++ |
| | cph1Δ::hisG/cph1Δ::hisG ade2::pDBI52 | sparse monolayer of cells | |
| | (URA3) | | |
| MC191 | ura3Δ::λimm434/ura3Δ::λimm434 | Functionally defective | +++ |
| | arg4::hisG/arg4::hisG his1::hisG/his1::hisG | hyphae | |
| | flo8::ARG4/flo8::HIS1 ade2::URA3/ADE2 | | |
| MC195 | ura3Δ::λimm434/ura3Δ::λimm434 | Robust 3D wild type | + |
| | arg4::hisG/arg4::hisG his1::hisG/his1::hisG | | |
| | flo8::ARG4/flo8::HIS1 ade2::URA3:FLO8- | | |
| | 2/ADE2 | | |
| MC245 | ura3Δ::λimm434/ura3Δ::λimm434 | Robust 3D wild type | ++ |
| | arg4::hisG/arg4::hisG his1::hisG/his1::hisG | | |
| | flo8::ARG4/FLO8 ade2::URA3/ADE2 | | |
| | HIS::his/his | | |
| DAY185 | Δura3::λimm434/Δura3::λimm434 | Robust 3D wild type | ++ |
| | arg4::hisG/arg4::hisG/pARG4-URA3 | | |
| | his1::hisG/his1::hisG/pHIS1 | | |
| DAY286 | Δura3::λimm434/Δura3::λimm434 | Robust 3D wild type | ++ |
| | arg4::hisG/arg4::hisG/pARG4-URA3 | | |
| | his1::hisG/his1::hisG | | |
| GKO443 | Δura3::λimm434/Δura3::λimm434arg4::his | Biofilm defect; decreased | ++ |
| | G/arg4::hisG his1::hisG/his1::hisG | biomass | |
| | suv3::Tn7-UAU1/suv3::Tn7-URA3 | | |
| GKO798 | Δura3::λimm434/Δura3::λimm434arg4::his | Biofilm defect; decreased | ++ |
| | G/arg4::hisG his1::hisG/his1::hisG | biomass | |
| | kem1::Tn7-UAU1/kem1::Tn7-URA3 | | |
| GKO814 | Δura3::λimm434/Δura3::λimm434arg4::his | Biofilm defect; decreased | ++ |
| | G/arg4::hisG his1::hisG/his1::hisG | biomass | |
| | nup85::Tn7-UAU1/nup85::Tn7-URA3 | | |
| GKO9 | Δura3::λimm434/Δura3::λimm434arg4::his | Biofilm defect; decreased | ++ |
| | G/arg4::hisG his1::hisG/his1::his-G | biomass | |
| | mds3::Tn7-UAU1/mds3::Tn7-URA3 | | |
| CJN702 | Δura3::λimm434/Δura3::λimm434arg4::his | Functionally defective | ++ |
| | G/arg4::hisG his1::hisG::pHIS1/his1::hisG | hyphae | |
| | bcr1::ARG4/bcr1::URA3 | | |
| CJN698 | Δura3::λimm434/Δura3::λimm434arg4::his | Robust 3D wild type | ++ |
| | G/arg4::hisG his1::hisG::pHIS1- | | |
| | BCR1/his1::hisG bcr1::ARG4/bcr1::URA3 | | |

¹+++ indicates 1-2% survival
²++ indicates 0.1-1% survival
³+ indicates 0.05-0.1% survival

### Example 2

### High Throughput Screening for Miconazole Potentiators

Given that known antifungals are inactive against persisters (with the exception of high levels of chlorhexidine), a screen was developed to identify potential persister compounds that in combination with a conventional antifungal agent would disable persister formation and eradicate infection. Specifically, a screen was developed using C. albicans cells treated with miconazole at subinhibitory concentrations, to which candidate potentiator compounds were added. Biofilms were grown in microtiter plates and the reduction of alamar blue was used as a quantitative readout. Alamar blue is reduced by live cells and produces a fluorescent indicator, which can be detected visually as a color change from blue to red. This primary screen did not discriminate between directly acting compounds and those that potentiate miconazole. Subsequent validation of primary hits as described below allowed identification of synergistically-acting compounds.

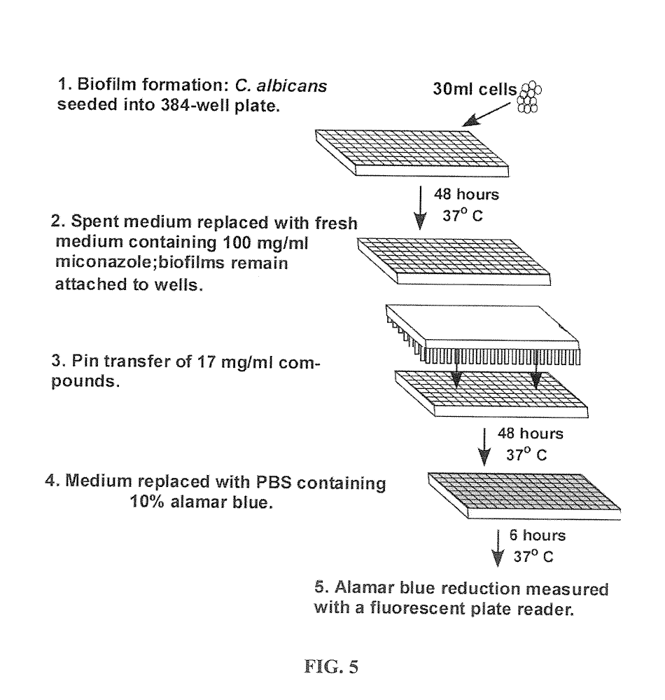

FIG. 5 schematically illustrates the biofilm high throughput screen (HTS) for potentiators of miconazole. Before screening for potentiators, the robustness of the screen was tested under control conditions to derive a Z′ factor. C. albicans was inoculated into RPMI 1640 medium and dispensed at 30 μl per well into a 384 well plate. After 48 hrs of incubation at 37° C., the medium was replaced with fresh medium containing 100 μg/ml miconazole (negative control) or a combination of 100 μg/ml miconazole and 50 μg/ml chlorhexidine (positive control). After an additional 48 hrs of incubation at 37° C., the medium was replaced with PBS containing 10% alamar blue. The Z factor was calculated by measuring the change in fluorescence produced by the reduction of alamar by C. albicans cells using a fluorescence plate reader by using the following formula:

Z′=1−(3SD⁺+3SD⁻)/(Ave⁺−Ave⁻)
where:
   SD⁺=positive control standard deviation;
   SD⁻=negative control standard deviation;
   Ave⁺=positive control average; and
   Ave⁻=negative control average

A Z′ of ≧0.5 indicates an effective screen, with 1.0 being the theoretically maximal value. A Z′ of 0.80 was calculated for the control experiment. The HTS was then performed to screen for potentiators, as depicted in FIG. 5.

First, biofilms were formed by seeding 30 μl of C. albicans (OD₆₀₀=0.1) in RPMI 1640 medium into 384-well plates. The plates were incubated for 48 hrs at 37° C. and then the medium was replaced with fresh medium containing 100 μg/ml miconazole. Test compounds from the chemical libraries described below were pin transferred at a final concentration of 17 μg/ml into individual wells of the microtiter plates. Biofilms were incubated for an additional 48 hrs and the medium was replaced with PBS containing 10% alamar blue. Plates were incubated at 37° C. for an additional 6 hrs, and alamar blue reduction was measured with a fluorescent plate reader with an excitation of 544 and an emission at 590 nm, respectively.

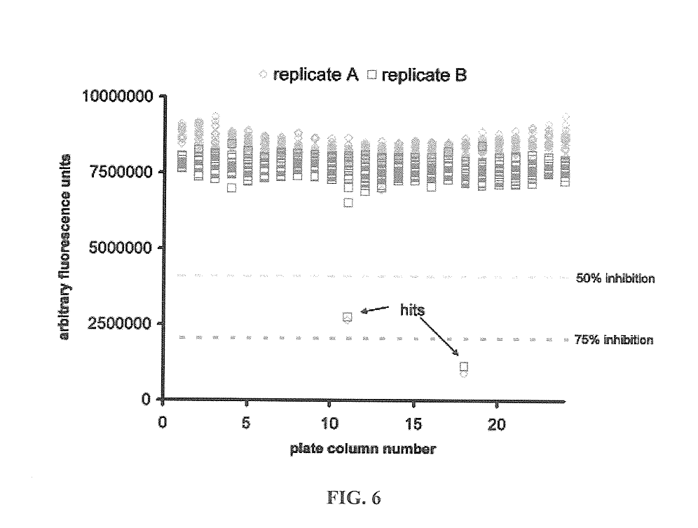

Approximately 70,000 compounds were screened in duplicate, producing 6 strong hits (exhibiting an inhibition of alamar blue reduction of greater than about 75%) and 52 medium hits (exhibiting an inhibition of between about 50% and about 75%) which were examined further. FIG. 6 shows the results from a representative compound platescreened in duplicate (plates A and B). The overall hit rate of the screen was 0.47%. The results of the screen are depicted in Table 2.

TABLE 2
Summary of HTS for Miconazole Potentiators

| | | | | | | Hit Rate |
|---|---|---|---|---|---|---|
| Library Name | # Screened | S | M | W | Total | (%) |
| Asinex 1 | 12,378 | | 1 | 12 | 13 | 0.11 |
| ChemBridge 3 | 8448 | | 5 | 42 | 47 | 0.56 |
| ChemDiv 3 | 11,968 | 1 | 3 | 1 | 5 | 0.04 |
| ChemDiv 4 | 1056 | | | 2 | 2 | 0.19 |
| Enamine 2 | 26,224 | 2 | 25 | 148 | 175 | 0.67 |
| Maybridge 5 | 3212 | | | 2 | 2 | 0.06 |
| Commercial Total | 63,286 | 3 | 34 | 207 | 244 | 0.39 |
| NINDS Custom | 1040 | | 1 | 10 | 11 | 1.06 |
| Collection 2 | | | | | | |
| BIOMOL ICCB Known | 480 | 2 | | 9 | 11 | 2.29 |
| Bioactives2 - High Conc. | | | | | | |
| BIOMOL ICCB Known | 480 | 1 | 1 | 1 | 3 | 0.63 |
| Bioactives1 - Med Conc. | | | | | | |
| Prestwick1 Collection | 1120 | | 8 | 9 | 17 | 1.52 |
| Bioactive Total | 3120 | 3 | 10 | 29 | 42 | 1.35 |
| NCDDG 1 | 380 | | 1 | 2 | 3 | 0.79 |
| ICBG 2 - Fungal Extracts | 460 | | 1 | 5 | 6 | 1.30 |
| ICBG 4 - Fungal Extracts | 704 | | 2 | 12 | 14 | 1.99 |
| Starr Foundation | 1000 | | 4 | 10 | 14 | 1.40 |
| Extracts 2 | | | | | | |
| Extract Total | 2544 | 0 | 8 | 29 | 37 | 1.45 |
| Summary | 68,950 | 6 | 52 | 265 | 323 | 0.47 |

The hits were verified for activity and then examined for their ability to kill biofilms in the presence of miconazole (100 μg/ml), by measuring colony count. Five compounds, including AC17, were able to kill biofilms in combination with miconazole, but not alone.

### Example 3

### In Vitro Validation of AC17

AC was subjected to in vitro evaluation of potency, toxicity, and ability to eradicate biofilms of high-persister mutants.

Potency

The lowest concentration of AC17 necessary for biofilm killing and persister eradication in the presence of miconazole was determined. Wild type mature C. albicans biofilms were challenged with AC17 for 48 hrs. Biofilms were washed, scraped, resuspended in PBS, vortexed for 30 secs and plated for colony count. Miconazole was present at 100 μg/ml.

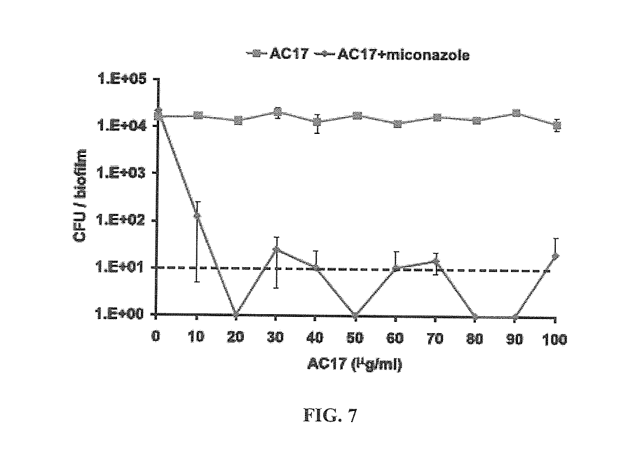

AC17 had strong potentiation activity against biofilm (FIG. 7) and did not have activity alone either against biofilms or growing cells (MIC>512 μg/ml).

Toxicity

An in vitro cytotoxicity assay was performed with primary human fibroblast cells IMR-90. Fibroblasts were grown at 37° C., 5% CO₂, in 10% FBS-DMEM, and seeded at 10⁵ cells per well into a 96-well flat bottom plate. Cells were incubated for 48 hrs to reach 70% confluence, and the compounds were added at a two-fold serial dilution in fresh growth medium. Cells were incubated for 24 hrs, and the medium containing the compounds was replaced with fresh growth medium. Fibroblasts were incubated for an additional 18 hrs, and cell viability was determined by alamar blue reduction. Using the alamar blue readout, the concentration of drug reducing viability by more than 50% (EC₅₀) was determined and used to calculate the therapeutic index (EC₅₀/MFCbiofilm, where MFCbiofilm is the minimal concentration at which AC17 causes biofilm eradication in the presence of miconazole). The EC₃₀, MFCbiofilm, and therapeutic indexes for AC17 were 250 μg/ml, 20 μg/ml, and 12.5, respectively. Cytotoxicity of AC17 was also tested in the presence of miconazole. The EC₅₀ of miconazole, alone, was determined to be 16 μg/ml. The addition of 16 μg/ml miconazole did not increase the cytotoxic effect for AC17.

Eradication of Bio Films of High-Persister Mutants

Periodic application of a high concentration of a bactericidal antibiotic leads to selection for high-persister “hip” mutants in E. coli in vitro. To determine whether a similar selection for hip mutants occurs in vivo, a collection of 131 clinical isolates of C. albicans was tested for their persister levels. These strains were obtained from patients who developed oral candidiasis as a result of anticancer chemotherapy. The patients were treated daily with topical chlorhexidine.

Biofilms were prepared from C. albicans clinical isolates in microtiter plates (as described in Example 1) and challenged with 100 μg/ml amphotericin B or 100 μg/ml chlorhexidine. Strains 1-6 were from patients with persistent candidiasis (group 1), and strains 7-15 were from patients whose infection resolved within 3 weeks (group 2).

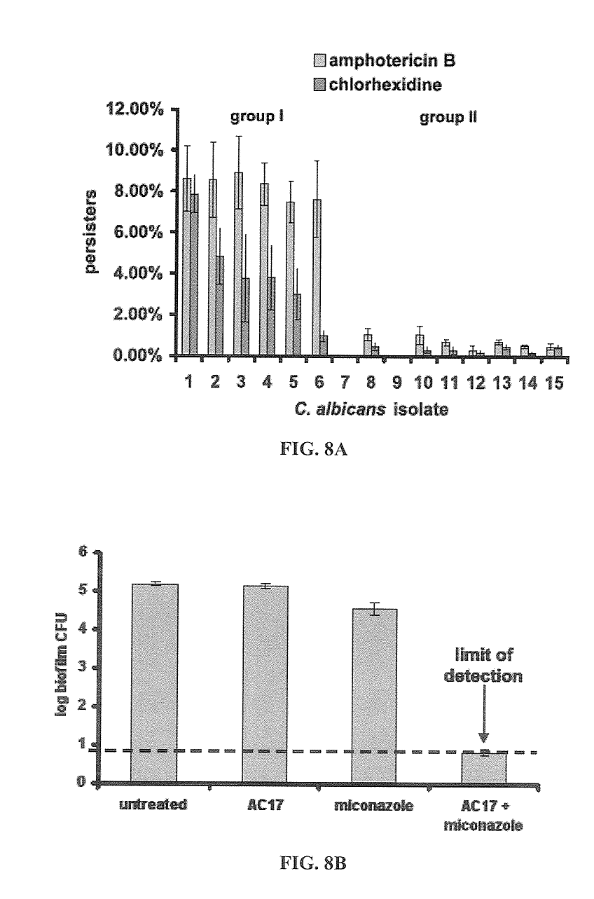

As shown in FIG. 8A, a considerable number of isolates had increased levels of surviving persisters. The MIC of these strains for amphothericin B and chlorhexidine was unchanged (1 μg/ml and 4 μg/ml, respectively), indicating that these were not resistant mutants, but rather hip mutants with increased drug tolerance. The only hip mutants came from patients whose disease failed to resolve within 3 weeks of treatment (FIG. 8A). Thus, these findings link microbial persisters with clinical manifestation of disease, suggesting that recalcitrance may be due to persister

Given that difficult-to-treat cases were linked to hip mutants of C. albicans the activity of AC17 was tested against those pathogens. Biofilms were grown from hip strains and challenged with AC17 (100 μg/ml), miconazole (100 μg/ml) or a combination of the two. As shown in FIG. 8B, the combination of miconazole and AC17 eradicated the biofilms.

### Example 4

### AC17 Inhibition of Biofilm Formation

As described above, AC17 was found to kill Candida biofilms in combination with miconazole. Whether AC17 had a direct effect on biofilm formation was determined.

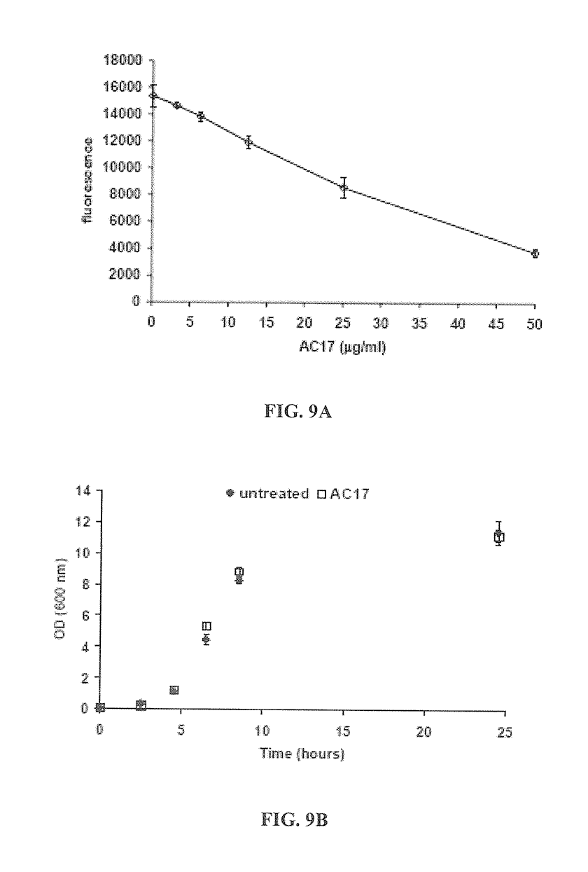

C. albicans cellular suspensions were adjusted to an OD₆₀₀ of 0.1 in RPMI medium according to standard biofilm formation protocols and various concentrations of AC17 were added from a 10 mg/ml stock solution. 100 μl aliquots were made into wells of flat-bottom microtiter plates (CoCostar 3370) which were incubated for 24 hr at 37° C. on a microtiter plate shaker (Lab-Line Instruments; model 4625) at approximately 100 rpm to allow for biofilm formation. After 24 hrs, biofilms were washed three times with sterile PBS to remove non-adherent cells and AC17. Biofilm metabolic activity was measured by adding 100 μl 10% alamar blue to wells and incubating at 37° C. for 2 hrs. Fluorescent intensity of biofilm metabolic activity was measured by reading plates in a fluorescent spectrophotometer with excitation at 544 nm and emission at 590 nm. As shown in FIG. 9A, AC17 inhibited the ability of cells to form wild type biofilms in a dose dependent manner.

To verify that AC17 had activity that was specific to the biofilm, a growth curve in YPD medium was performed on AC17 treated and untreated cells. Strain CAF2-1 was grown ON in YPD medium and diluted in fresh YPD medium and YPD medium containing 30 μg/ml AC17. Optical density at 600 nm was measured using a spectrophotometer at various time points for 24 hrs to generate a growth curve. No differences in growth rate was detected in AC17 treated yeast cells that were grown in YPD medium compared to the untreated control (see FIG. 9B). Cell size and morphology also appeared completely normal based on microscopic analysis.

### Example 5

### AC17 Inhibition of Hyphal Elongation

To better understand how AC17 inhibited biofilm formation and did not have direct growth inhibitory activity, the effect of AC17 on filamentous growth was determined. Microscopy was used to analyze whether AC17 specifically inhibited yeast to hyphae transition or prevented hyphal elongation.

C. albicans cells from ON cultures grown in YPD medium were diluted into RPMI medium to an OD₆₀₀ nm of 0.2. 1 ml of the cell suspensions were aliquoted into 15 ml culture tubes and appropriate concentrations of AC17 were added to the test tubes from a 10 mg/ml stock solution dissolved in RPMI medium. Tubes were incubated at 37° C. for 12 hrs in a 200 rpm shaking incubator. After 12 hrs, samples from the tubes were wet mounted and photographed using a Zeiss Axioskop 2 microscope with AxioCam black and white CCD camera (Carl Zeiss). Hyphal lengths were quantified using Axiovision Rel. 4.5 software by identifying yeast cells from which single germ tubes originated and measuring the distance from the beginning to the end of the hyphal tip. Measurements from 90 cells per treatment were averaged and the experiment was performed with biological duplicate.

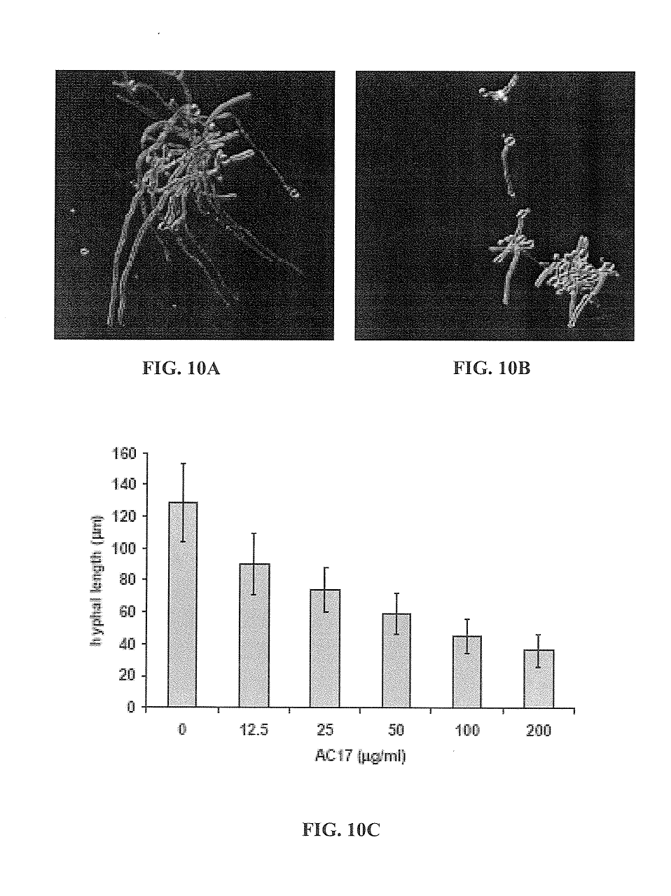

Microscopy revealed that untreated cells (FIG. 10A) had longer hyphae compared to AC17 treated cells (FIG. 10B). When the lengths of individual hyphae were measured after 12 hrs of growth in RPMI 1640 medium containing AC17, a concentration dependent attenuation of hyphal elongation was detected (FIG. 10C). Treatment with 50 μg/ml of AC17 caused a two fold reduction in hyphal length and 12.5 μg/ml was sufficient to cause a significant difference in hyphae length compared to untreated controls. Microscopy also revealed that AC17 did not inhibit yeast to hyphae transition, since germ tubes were present on almost every cell (FIG. 10B).

### Example 6

### AC17 Prevention of Invasive Growth

Invasive growth by Candida mediates pathogenesis and the establishment of infection in vivo. The ability of AC17 to prevent invasion into solid medium was assayed. Several genetic pathways activate filamentation and invasive growth. For example, transcription factor CZF1 mediates invasion in embedded growth conditions, while mutation of CPH1 results in defective filamentous growth in certain media, but displays a mild defect within agar. Thus, the ability of AC17 to inhibit invasion into a variety of media under several conditions was tested.

Invasive growth was determined by spread plating approximately 100 C. albicans cells of an ON culture grown in YPD medium at 30° C. onto the surface of Lee's, Spider and YPS agar medium. Lee's medium contained 5.0 g (NH₄)₂SO₄, 0.2 g MgSO₄, 2.5 g K₂HPO₄ (anhydrous), 5.0 g NaCl, 12.5 g mannitol, 0.5 g L-alanine, 1.3 g L-leucine, 1.0 g L-lysine, 0.1 g L-methionine, 0.0714 g L-ornithine, 0.5 g L-phenylalanine, 0.5 g L-proline, 0.5 g L-threonine, 0.001 g biotin, and 15 g agar per 1000 ml of distilled water. Spider medium contained 1% nutrient broth (Difco), 1% mannitol, 1.35% agar, and 2 g/L KH₂PO₄. YPS medium contained 1% bacto peptone (Difco), 0.5% yeast extract (Difco), 10.5% agar, and 2% sucrose (Fluka). Invasive growth was also determined for cells that were embedded in YPD agar medium, Approximately 100 cells were spread onto YPD agar and a thin layer of molten YPD agar was poured over the cells. AC17 was added to the media at the appropriate concentrations from 10 or 50 mg/ml stock solutions of AC17 dissolved in water. The lowest concentration of AC17 required to prevent invasive growth was determined by making 2 fold dilutions of AC17 in Spider agar. Lee's. Spider and YPD agar plates were incubated at 37° C. for 5-7 days. YPS agar plates were incubated at 25° C. for 5-7 days. Invasive growth was determined by visual inspection, and photographs of individual colonies were taken using a AxioCam black and white CCD camera and a Zeiss Discovery V12 stereoscope.

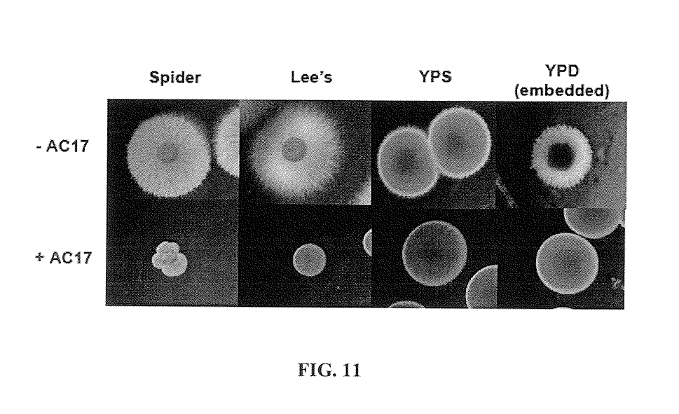

As shown in FIGS. 11A-11C, AC17 prevented invasion into Lee's, Spider, and YPS medium when yeast cells were plated on top of the agar. AC17 also prevented invasion under embedded growth conditions when cells were seeded within molten YPD agar medium (FIG. 11D). Concentrations of AC17 as low as 3 μg/ml prevented invasive growth into Lee's, Spider, and YPS medium, suggesting AC17 is a potent inhibitor of invasion. AC17 inhibited invasion under all growth and media conditions that were tested, suggesting AC17 targets a factor common to most or all known invasion pathways.

### Example 7

### AC17 Targets UME6 Pathway

UME6 is known to be a transcription factor required for the maintenance of hyphal growth. UME6 may be a putative target for AC17, since a Δume6 mutation results in a shortened hyphae phenotype similar to AC17 treated cells. To test whether AC17 targets the UME6 pathway, AC17 treated cells were compared to UME6 mutants.

Wild type C. albicans cells and C. albicans strains UZ24 (ume6Δ::CmLEU2/UME6 his1Δ/his1Δ), UZ43 (ume6Δ::CdHIS1/ume6Δ::CmLEU2), and UZ149 ((ADH1/adh1::Ptet-UME6 (PACT1-CaSAT1) UME6/UME6) (Zeidler et al., FEMS Yeast Res. 9:126-142 (2009)) were grown as described above. Yeast cells were grown in the presence of 100 μg/ml AC17 and aliquots of RPMI-cell suspensions were placed into 15 ml culture tubes. AC17 was added to culture tubes when appropriate so the final concentrations were 100 μg/ml. 20 μg/ml doxycycline was added to UZ149 to induce UME6 expression. The tubes were incubated at 37° C. in a shaking incubator at 200 RPM for approximately 18 hrs. Samples from culture tubes were wet mounted on microscope slides and photographed under 40× magnification using a Zeiss Axioskop 2 microscope with Axiovision Rel. 4.5 software and AxioCam (Carl Zeiss) black and white CCD camera.

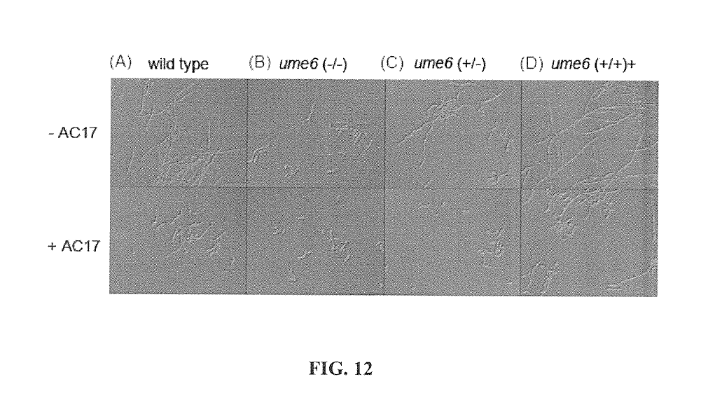

FIG. 12 shows the effects of AC17 on wild type (A), Δ/Δ ume6 (B), ume6 heterozygote (C), and UME6 overexpression (D), compared to untreated control cells of each strain. AC17 blocked hyphal elongation in the wild type, ume6 heterozygote and overexpression strains, while it had no effect on Δ/Δ ume6. AC17-treated ume6 heterozygote cells also closely resembled untreated Δ/Δ ume6 cells. These results indicate that AC17 targets UME6 or its pathway.

The ability of AC17 treatment to cause hyphae reversion of growth back to yeast when UME6 was overexpressed under non-filamenting conditions was tested. Yeast strain UZ149 was grown ON in YPD liquid medium at 37° C. with and without 10 μg/ml doxycycline to induce hyphal growth. After ON incubation at 37° C. and 200 rpm, yeast and hyphae cells were harvested by centrifugation, washed twice in sterile PBS, and diluted 1:500 in fresh YPD medium. The YPD medium contained either 10 μg/ml doxycycline, a combination of 10 μg/ml doxycycline and 100 μg/ml AC17, or no additional drugs. Culture tubes were incubated an additional 18 hrs at 37° C. and 200 rpm. Samples from culture tubes were wet mounted on microscope slides and photographed under 40× magnification using a Zeiss Axioskop 2 microscope with Axiovision Rel. 4.5 software and AxioCam (Carl Zeiss) black and white CCD camera.

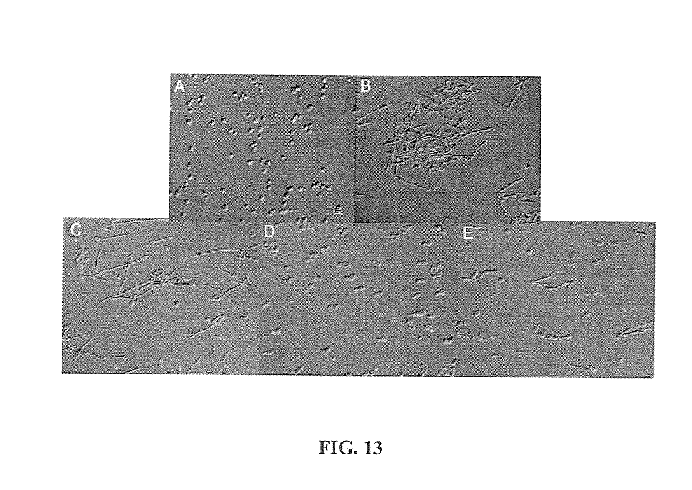

When the UME6 overexpression strain UZ149 was grown ON in YPD medium at 37° C., only yeast cells were present (FIG. 13A). When UME6 inducer doxycycline (25 μg/ml) was added to the YPD medium, hyphal growth resulted, as indicated by the presence of germ tubes (FIG. 13B). When cells were diluted and grown in the continued presence of doxycycline, the hyphal states were maintained (FIG. 13C). However, removing doxycycline by washing resulted in reversion to yeast growth (FIG. 13D). Further, the addition of AC17 to doxycycline-treated cells also resulted in reversion to predominantly yeast growth (FIG. 13E). While there were a few germ tubes present, the AC17-treated cells mimicked the effects of doxycycline removal, in contrast to cells grown continuously in the presence of doxycycline. These germ tubes may be the result of the high level of artificial Ume6 over expression and incomplete UME6 inhibition by AC17 under these conditions, AC17 appeared to target the UME6 pathway, since AC17 blocked the effects of UME6 overexpression.

### Example 8

### Treatment of Fungal Infections on Catheters with AC17 and Miconazole

To treat a catheter that is infected with C. albicans, a catheter lock solution containing 2% miconazole and 1% AC17 is prepared. The catheter is treated by locking the catheter lumen and administering the solution for 2 hrs a day for 7 days, while the catheter is not in use. It is then rinsed before use. Treatment of the catheter with the miconazole/AC17 solution eliminates the biofilm and persisters, and allows for continued use of the catheter.

### Example 9

### Treatment of Oral Candidiasis with AC17 and Clotrimazole

To treat oral candidiasis in a subject, lozenges containing 10 mg clotrimazole and 5 mg AC are prepared. The lozenges are administered 5 times a day for 7 days. The use of lozenges containing a combination of AC17 and clotrimazole eliminates biofilms and persisters, and prevents the recurrence of disease.

### Example 10

### Treatment of Vaginitis with AC17 and Miconazole

To treat a vaginal yeast infection caused by Candida in a subject, 1% AC17 is added to a cream containing 2% miconazole, hydrogenated vegetable oil base, benzoic acid, cetyl alcohol, isopropyl myristate, polysorbate 60, potassium hydroxide, propylene glycol, purified water, and stearyl alcohol (e.g., Monistat®cream). The cream is administered to the affected area of the vagina once a day for seven days. The addition of AC17 to miconazole-containing cream increases efficacy of the miconazole and prevents recurrent disease.

### EQUIVALENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of inhibiting the growth of, or killing, a fungus, the method comprising contacting the fungus with an effective amount of:
(a) an antifungal agent; and
(b) one or more potentiator compounds of Formula I: 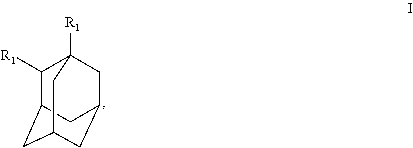
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl,
wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or NH₂, thereby inhibiting the growth of, or killing, the fungus.

2. The method of claim 1, wherein the potentiator compound potentiates the activity of the antifungal agent.

3. The method of claim 1, wherein the potentiator compound is not an antifungal compound.

4. A method of inhibiting the growth of, or killing, a fungus, the method comprising contacting the fungus with an effective amount of:
(a) an antifungal agent; and
(b) one or more potentiator compounds of Formula Ia: 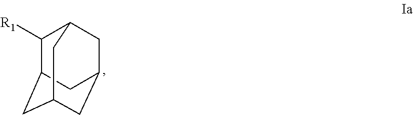
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl,
wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or NH₂,
thereby inhibiting the growth of, or killing, the fungus.

5. The method of claim 4, wherein the compound is

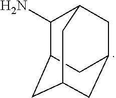

6. The method of claim 4, wherein the compound is

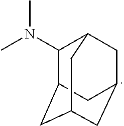

7. The method of claim 4, wherein the compound is

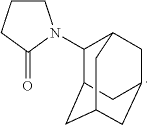

8. The method of claim 4, wherein the compound is

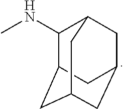

9. The method of claim 4, wherein the potentiator compound enhances the activity of the antifungal agent.

10. The method of claim 4, wherein the potentiator compound is not an antifungal compound.

11. A method of inhibiting the growth of, or killing, a fungus, the method comprising contacting the fungus with:
(a) an antifungal agent; and
(b) one or more potentiator compounds of Formula Ib: 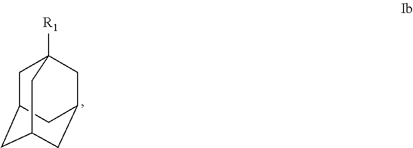
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl,
wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or NH₂.
thereby inhibiting the growth of, or killing, the fungus.

12. The method of claim 11, wherein the compound is

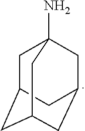

13. The method of claim 11, wherein the compound is

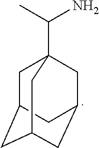

14. The method of claim 11, wherein the potentiator compound potentiates the activity of the antifungal agent.

15. The method of claim 11, wherein the potentiator compound is not an antifungal compound.

16. A method of treating a fungal infection in a subject in need thereof, the method comprising administering to the subject an effective amount of:
(a) an antifungal agent; in combination with
(b) an effective amount of one or more potentiator compounds of Formula I: 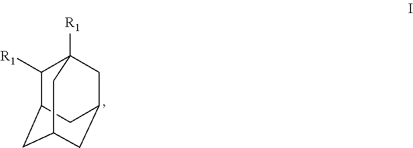
or pharmaceutically acceptable salts, hydrates, and solvates hereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or NH₂,
thereby treating the fungal infection.

17. The method of claim 16, wherein the potentiator compound potentiates the activity of the antifungal agent.

18. The method of claim 16, wherein the potentiator compound is not an antifungal compound.

19. A method of treating a fungal infection in a subject in need thereof, the method comprising administering to the subject an effective amount of
(a) an antifungal agent; in combination with
(b) an effective amount of one or more potentiator compounds of Formula Ia: 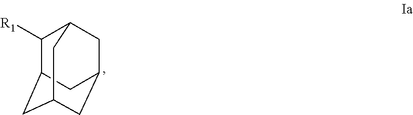
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl,
wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or NH₂,
thereby treating the fungal infection.

20. The method of claim 19, wherein the compound is

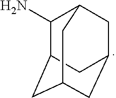

21. The method of claim 19, wherein the compound is

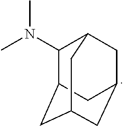

22. The method of claim 19, wherein the compound is

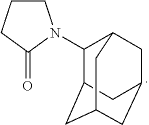

23. The method of claim 19, wherein the compound is

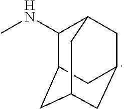

24. The method of claim 19, wherein the potentiator compound potentiates the activity of the antifungal agent.

25. The method of claim 19, wherein the potentiator compound is not an antifungal compound.

26. A method of treating a fungal infection in a subject in need thereof, the method comprising administering to the subject an effective amount of:
(a) an antifungal agent; in combination with
(b) an effective amount of one or more potentiator compounds of Formula Ib: 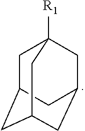
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl,
wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or NH₂,
thereby treating the fungal infection.

27. The method of claim 26, wherein the potentiator compound enhances the activity of the antifungal agent.

28. The method of claim 26, wherein the potentiator compound is not an antifungal compound.

29. A method of treating or preventing relapsing vaginitis in a subject in need thereof, the method comprising administering to the subject an effective amount of:
(a) miconazole, in combination with,
(b) an effective amount of potentiator Compound 1: 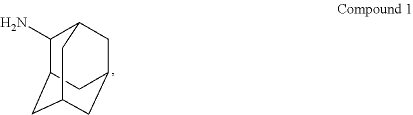
thereby treating the relapsing vaginitis in the subject.

30. A method of treating or preventing oral candidiasis in a subject in need thereof, the method comprising administering to the subject an effective amount of
(a) miconazole; in combination with,
(b) an effective amount of potentiator Compound 1: 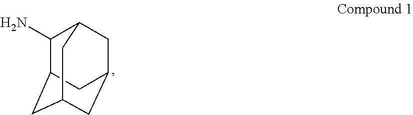
thereby treating the oral candidiasis in the subject.

31. A method of treating a fungal infection of a medical device, the method comprising administering to the device an effective amount of:
(a) miconazole, in combination with,
(b) an effective amount of potentiator Compound 1: 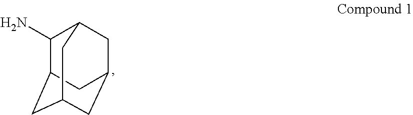
thereby treating the fungal infection.

32. A method of inhibiting the growth of, or killing, a fungus, the method comprising contacting the fungus with: 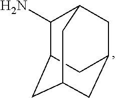
thereby inhibiting the growth of, or killing, the fungus.

33. A method of treating a fungal infection in a subject in need thereof, the method comprising administering to the subject an effective amount of: 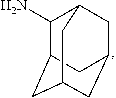
thereby treating the fungal infection.

34. A method of treating or preventing relapsing vaginitis in a subject in need thereof, the method comprising administering to the subject an effective amount of potentiator Compound 1: 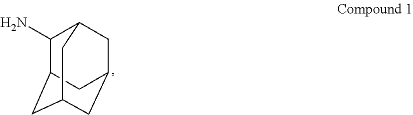
thereby treating the relapsing vaginitis in the subject.

35. A method of treating or preventing oral candidiasis in a subject in need thereof, the method comprising administering to the subject an effective amount of potentiator Compound 1: 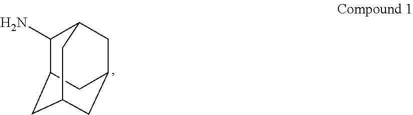
thereby treating the oral candidiasis in the subject.

36. A method of treating a fungal infection of a medical device, the method comprising administering to the device an effective amount of potentiator Compound 1: 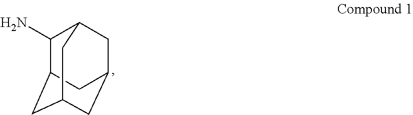
thereby treating the fungal infection of the device.

37. A method of inhibiting the growth of, or killing, a C. albicans fungus, the method comprising contacting the fungus with an effective amount of:
(a) an antifungal agent; and
(b) one or more potentiator compounds of Formula I: 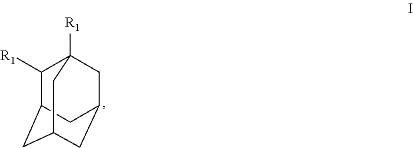
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl,
wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or NH₂,
thereby inhibiting the growth of, or killing, the fungus.

38. A method of inhibiting the growth of, or killing, a C. albicans fungus, the method comprising contacting the fungus with an effective amount of:
(a) an antifungal agent; and
(b) one or more potentiator compounds of Formula Ia: 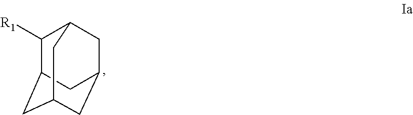
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl,
wherein the aryl is optionally substituted, or a five-membered heteroaryl, optionally substituted with alkyl, halogen. OH, or NH₂, or 5- or 6-membered heterocycle, optionally substituted with alkyl, halogen, OH, NH₂, or a carbonyl, or alkyl, optionally substituted with alkyl, halogen, OH, or NH₂,
thereby inhibiting the growth of, or killing, the fungus.

39. A method of inhibiting the growth of or killing, a C. albicans fungus, the method comprising contacting the fungus with:
(a) an antifungal agent; and
(b) one or more potentiator compounds of Formula Ib: 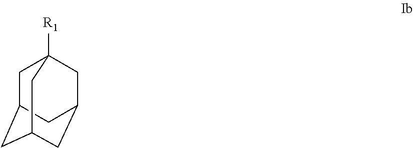
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl,
wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or
thereby inhibiting the growth of, or killing, the fungus.

40. A method of treating a C. albicans fungal infection in a subject in need thereof, the method comprising administering to the subject an effective amount of:
(a) an antifungal agent; in combination with
(b) an effective amount of one or more potentiator compounds of Formula I: 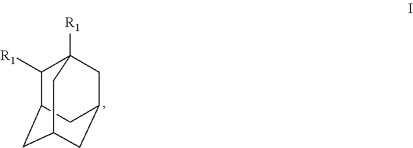
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl, wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or NH₂,
thereby treating the fungal infection.

41. A method of treating a C. albicans fungal infection in a subject in need thereof, the method comprising administering to the subject an effective amount of:
(a) an antifungal agent; in combination with
(b) an effective amount of one or more potentiator compounds of Formula Ia: 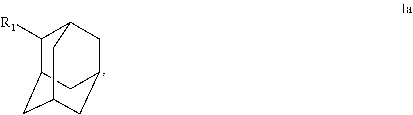
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl,
wherein the alkyl is optionally substituted, —NH(S(O)₂)aryl,
wherein the aryl is optionally substituted, or a five-membered heteroaryl, optionally substituted with alkyl, halogen, OH, or NH₂, or 5- or 6-membered heterocycle, optionally substituted with alkyl, halogen, OH, NH₂, or a carbonyl, or alkyl, optionally substituted with alkyl, halogen, OH, or NH₂,
thereby treating the fungal infection.

42. A method of treating a C. albicans fungal infection in a subject in need thereof, the method comprising administering to the subject an effective amount of:
(a) an antifungal agent; in combination with
(b) an effective amount of one or more potentiator compounds of Formula Ib: 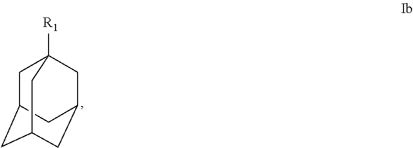
or pharmaceutically acceptable salts, hydrates, and solvates thereof,
wherein each R₁ is independently —OH, —OC(O)H, —OC(O)alkyl, —NH₂, —NH-alkyl, —N(alkyl)₂, —NH-aminoacid, —NHC(O)alkyl, —NHC(O)aryl, —NH(S(O)₂)alkyl,
wherein the alkyl is optionally substituted, —NH(S(O)₂)aryl,
wherein the aryl is optionally substituted, or a five-membered heteroaryl, optionally substituted with alkyl, halogen. OH, or NH₂, or 5- or 6-membered heterocycle, optionally substituted with alkyl, halogen, OH, NH₂, or a carbonyl, or alkyl, optionally substituted with alkyl, halogen, OH, or NH₂,
thereby treating the fungal infection.
